(19) United States
(12) Patent Application Publication
(10) Pub. No.: US 2023/0310573 A1
(43) Pub. Date: Oct. 5, 2023

(54) Stabilized Vaccine Compositions

(71) Applicant: Janssen Vaccines & Prevention B.V., Leiden (NL)

(72) Inventors: Tina RITSCHEL, Oegstgeest (NL); Richard VOORZAAT, Oude Wetering (NL); Lucy RUTTEN, Rijnsburg (NL); Mandy Antonia Catharina JONGENEELEN, Leiden (NL); Tim Hugo Maria JONCKERS, Heist-op-den-Berg (BE); Dirk André Emmy ROYMANS, Turnhout (BE); Johannes Petrus Maria LANGEDIJK, Amsterdam (NL)

(21) Appl. No.: 17/995,158

(22) PCT Filed: Apr. 1, 2021
(86) PCT No.: PCT/EP2021/058601

§ 371 Date: Sep. 30, 2022

(30) Foreign Application Priority Data
Apr. 2, 2020 (EP) 20167718.4

(51) Int. Cl. A61K 39/145; A61P 31/12

(52) U.S. Cl. CPC A61K 39/145; A61P 31/12; A61K 2039/545

(57) ABSTRACT

The present invention relates to vaccine composition comprising an immunologically effective amount of a viral fusion protein antigen, such as an RSV pre-fusion F protein, and a stabilizing amount of an antiviral compound, and to methods for preparing such vaccine compositions.

### CROSS-REFERENCE TO RELATED APPLICATIONS

This application is a Section 371 of International Application No. PCT/EP2021/058601, filed on Apr. 1, 2021, that published in the English language on Oct. 7, 2021, under International Publication No. WO 2021/198413 A1, that claims priority to European Patent Application No. 20167718.4, filed on Apr. 2, 2020. Each disclosure is incorporated herein by reference in its entirety.

### REFERENCE TO SEQUENCE LISTING SUBMITTED ELECTRONICALLY

This application contains a sequence listing that is submitted electronically via EFS-Web as an ASCII formatted sequence listing with a file name “205US1 Sequence Listing” and a creation date of Sep. 27, 2022, and having a size of 45 kb. The sequence listing is submitted via EFS-Web is part of the specification and is herein incorporated by reference in its entirety.

### INTRODUCTION

The invention relates to the field of medicine. The invention, in particular, relates to stabilized vaccine compositions, in particular stable vaccine compositions comprising recombinant pre-fusion class I fusion proteins, and to uses thereof.

### BACKGROUND

Enveloped viruses, such as the respiratory syncytial virus (RSV) or influenza viruses, enter cells by inducing fusion of viral and cellular membranes, a process catalyzed by a specialized membrane-fusion protein expressed on their surface. The fusion glycoproteins are present in a labile (metastable) form at the surface of infectious virions and are dynamic fusion machines that drive the membrane fusion by irreversible protein refolding from said metastable pre-fusion conformation to a stable post-fusion conformation.

The fusion proteins of enveloped viruses can be classified in different types based on the general irreversible folding mechanism they display to drive fusion of the virus with the target cell. Fusion proteins from unrelated viruses, such as the fusion (F) protein from Paramyxoviridae, the Retroviridae envelope protein, the Coronaviridae spike protein and Orthomyxovirideae Hemagglutinin (HA) protein and others, are classified as class I fusion proteins and refold from a labile pre-fusion state to a stable post-fusion state through a similar mechanism although they do not exhibit any significant sequence homologies. Structures have been determined for a variety of class I fusion proteins in pre-fusion conformation and post-fusion conformation providing insight into the mechanism of this complex fusion machine.

These structural studies have also shown that stable class I fusion proteins presenting the same antigenic sites as the labile wild-type proteins efficiently elicit potently neutralizing antibodies and thus are suitable for use as antigen in vaccines. When viral fusion proteins are used as immunogens in a vaccine their fusogenic function is not important. However, the mimicry of the immunogen to the native viral surface proteins is important to ensure that the vaccine induces cross reactive antibodies that can bind the virus. Indeed, the immunogenicity of protein vaccines depends (to a large extent) on the structural integrity of the key protein antigens, especially in relation to conformational epitopes (where antibodies are required to bind disparate regions of the polypeptide chain brought together by native folding). Irreversible conformational changes and irreversible aggregation thus can lead to a reduced efficacy of vaccines. Therefore, for development of said class I fusion proteins as robust efficacious vaccine immunogens it is desirable that these meta-stable fusion proteins are stably maintained in their pre-fusion conformation.

However, many protein vaccines are unstable to heat, or (long-term) storage at 4° C., or to freeze-thawing and thus can lose their potency upon storage. Improvement of (thermo)stability can solve cold-chain problems that for example may be faced in remote and poorer areas, and prolong the shelf life of the vaccine. A need thus exists to produce stable vaccine compositions with an increased (thermo)stability and/or an increased shelf life.

### SUMMARY OF THE INVENTION

The invention relates to vaccine compositions comprising viral fusion proteins, such as class I fusion proteins, as antigen, which vaccine compositions have improved (thermo)stability. The vaccine compositions of the invention can be stored under frozen or refrigerated conditions for extended periods of time and are suitable for use in clinical and commercial manufacturing. The invention also relates to methods of preparing the stabilized vaccine compositions.

In a first aspect, the invention relates to vaccine compositions comprising an immunologically effective amount of a viral fusion protein antigen, in particular a pre-fusion class I fusion protein, and a stabilizing amount of an antiviral compound.

The vaccine compositions of the present invention have an improved thermal stability and improved stability against agitation stress, thermal stress (e.g. freeze-thaw stress), stress by pH fluctuations and consequently an extended shelf life, as compared to previously disclosed compositions.

According to the present invention it has surprisingly been shown that by adding a stabilizing amount of an antiviral compound, such as a small molecule fusion inhibitor or viral entry inhibitor, to said vaccine compositions, the viral fusion protein remains stable in the pre-fusion conformation, even under stressful conditions, such as freeze-thawing cycles.

In a further aspect, the invention relates to methods for preparing a vaccine composition comprising a protein antigen, said method comprising admixing an immunologically effective amount of said protein antigen with a stabilizing amount of an antiviral compound.

In a particular aspect, the invention provides method for reducing aggregation of viral fusion proteins in a vaccine composition.

In another aspect, the invention provides method for reducing aggregation of viral fusion proteins in a vaccine composition after freeze-thawing of said vaccine composition.

In yet another aspect, the invention provides methods for preserving a vaccine comprising a protein antigen, which method comprises preparing a vaccine composition as described herein.

The invention further provides methods for stably maintaining a liquid vaccine composition comprising a protein antigen, the method comprising storing a vaccine composition as described herein at a temperature of 2-8° C. for at least 20 months.

### BRIEF DESCRIPTION OF THE FIGURES

The foregoing summary, as well as the following detailed description of the invention, will be better understood when read in conjunction with the appended drawings. It should be understood that the invention is not limited to the precise embodiments shown in the drawings.

FIG. 1: A. SEC analysis of a purified preF. B. SDS-PAGE analysis of RSV pre-fusion F protein (preF) under non-reducing (lane 2) and reducing (lane 3) conditions. The gel was stained with Coomassie Brilliant Blue. C. Purified preF was measured on Q Octet assay with CR9501 and CR9506 monoclonal antibodies. CR9501 binds site V and is specific for the pre-fusion conformation of RSV-F (Gilman et. al., Nat. Comm 2019). CR9506 binds site II and is specific for the pre-fusion conformation and post-fusion conformation of RSV F.

FIG. 2: Temperature stability analysis of PreF protein by Differential Scanning Fluorimetry (DSF). Difference in melting temperature (ΔTm₅₀) between pre-fusion F without and pre-fusion F with antiviral compound in ° C. Antiviral compounds used are indicated with Roman numerals I-VXI (table 1). The stabilizing compounds are added in three trimer:compound molar ratios: 1:3, 1:10 and 1:100. All stabilizing compounds were dissolved in DMSO. Same amounts of DMSO as used in the 1:3, 1:10 and 1:100 compositions were added to the protein as control samples.

FIG. 3: Analytical SEC of PreF trimer in phosphate buffer after slow freeze/thaw process without (n=6) (A) and with fusion inhibitor (trimer:compound molar ratios: 1:3) II (B) and III (C) (n=3). Peak before 6 minutes is aggregate and peak at 7 minutes is the trimer peak.

FIG. 4: Residual PreF trimer measured by analytical SEC with and without fusion inhibitors (Table 1) after slow freeze process in different formulation buffers. In FIG. 4A, four different formulation buffers are tested, i.e. AP1, FB12 and formulation 1 and 2. In B a large set of compounds at different ratios is tested in Formulation 1. In C an additional buffer (Formulation 5) at two different pH values (pH 7.0, formulation 5a and pH 8.3, formulation 5b) is tested. D shows results of formulation compositions 1,2 and AP1 without polysorbate (AP1-P). Depicted is the trimer content relative to the 4° C. control samples on the y-axis. Averaged data ±SD. Dotted line: 100% trimer, dashed line: 90% trimer.

FIG. 5: SEC analysis of preF protein in different formulation buffers with and without stabilizing compound III (trimer:compound molar ratios: 1:3 and 1:9) after 6 weeks stored at 37° C. (A) and at 40° C. (B). Retention time for aggregates is around 3.1 minutes and for trimer around 4.2 minutes. Six weeks stored samples at higher temperature are shown as dashed lines and are compared to sample kept at 4° C. indicated as black line.

FIG. 6: Impact of addition of stabilizing compound on RSV F expression in crude supernatant after transfection. For all three samples the same volume of supernatant was injected. Trimer content in supernatant was measured using SEC for RSV F stabilized in the prefusion conformation (preF; SEQ ID NO:1) (black solid line), unstabilized consensus RSV A F (dashed black line; SEQ ID NO: 2) and unstabilized consensus RSV A F (SEQ ID NO:2) expressed in the presence of compound III (compound concentration of 0.28 μM) (grey line).

FIG. 7: HIV-1 ConB-SOSIP was produced and purified as described (Rutten et. al., Cell Reports 2018). A. SEC analysis of a purified HIV-1 Env ConB-SOSIP. B. Melting temperature (Tm₅₀) with and without addition of entry inhibitor XVII (Table 2) added with a 50-fold molar excess to the purified Env ConB SOSIP and stored for a week at 4° C. C. Binding of broadly neutralizing antibodies (bNAbs) and non-broadly neutralizing antibodies (non-Nabs), measured with AlphaLISA

FIG. 8. (A) Melting temperature (Tm₅₀) of purified soluble HA proteins corresponding to Massachusetts/2/12 (left panel) and Colorado/06/2017 (right panel) with and without a 50-fold excess of influenza B entry inhibitor XXVII (Table 3). Tm₅₀ was measured in triplicates using DSF for protein without addition (black), DMSO only (grey) and DMSO plus inhibitor (white). (B) Analytical SEC analysis of soluble influenza B HA ectodomain (UFV 180933) in cell culture supernatant 4 days after transfection of ExpiHEK293F cells (dashed line) with addition of DMSO (dotted line) and addition with DMSO plus 30-fold excess of entry inhibitor (black line). Trimer peak at 4.0 minutes and monomer peak at 4.3 minutes.

FIG. 9: Analytical SEC analysis of soluble influenza A HA ectodomain (H1N1 A/Brisbane/59/2007) after 6 weeks at 40 degree Celsius (gray dashed line), with addition of DMSO (dotted gray line) and with addition of compound XXIV (black solid line). Retention time for trimer is around 4.1 minutes and for monomer around 4.5 minutes.

FIG. 10: Liquid stability. Trimer content of RSV preF protein after storage in different formulation buffers with and without stabilizing compound III at 37° C. after 9, 16 and 26 weeks based on SEC analysis. Ttrimer:compound molar ratios: 1:3 and 1:9. The trimer content is shown relative to a 4° C. control. Individual replicates (n=2), mean and error bars indicating the standard deviation are depicted.

FIG. 11. Dialysis excess of stabilizing compound III. After dialysis and slow freezing, samples were thawed on the bench and assessed for their (a) aggregates level by analytical SEC and (b) melting temperature by DSF (Day 0). Samples were then stored a 4° C. and aliquots were taken on Day 21 and Day 84 for additional DSF analysis. Individual replicates (n=5 for slow freeze; n=3 for DSF), mean and error bars indicating the standard deviation are depicted.

FIG. 12. UF/DF excess of stabilizing compound III. After UF/DF and slow freezing, samples were assessed for their (a) aggregation level by analytical SEC and (b) melting temperature by DSF (Day 0). Samples were then stored a 4° C. and aliquots were taken on Day 21 and Day 84 for additional DSF analysis. Individual replicates (n=5 for slow freeze; n=3 for DSF), mean and error bars indicating the standard deviation are depicted.

FIG. 13. Buffer exchange using different buffers & and excess stabilizing compound III. Samples that contained stabilizing compound III, were pre-incubated for 24 h at room temperature (RT). Samples were then dialyzed (preF against Formulation 2 without PS20 and AP1 buffers) or UF/DF-ed (preF against Formulation 1 without PS20 and preF+1:50 against Formulation 1 and 2 without PS20 and AP1) and subsequently, slow frozen (to −70° C. in 24 h). Then, samples were assessed for (a) aggregation level by analytical SEC, (b) melting temperature by DSF (Day 0). Individual replicates (n=5 for slow freeze; n=3 for DSF), mean and error bars indicating the standard deviation are depicted. (c) Quantification of stabilizing compound III after UF/DF. Samples that were previously UF/DF-ed and snap frozen were used to quantify the amount of FI left after buffer exchange. Quantification was performed using a LC-MS/MS. Plotted in the left Y axis the concentration from each sample. Right Y axis corresponds to the equivalence of stabilizing compound III molecules per trimer.

FIG. 14. RSV CL57 neutralizing antibody titers at day 42. Mice were immunized at day 0 and 28 and serum was collected at day 42. VNA titers were measured by firefly luciferase assay using the RSV CL57 strain and are expressed as the log 2 value of the IC90 titer. The black bars specify the mean of response within each group and the dotted line represents the lower limit of quantification (LLoQ).

FIG. 15. Statistical analysis of RSV CL57 neutralizing antibody titers. The comparisons between VNA titers in groups immunized with preF+ stabilizing compound III with titers in the groups immunized with preF protein alone were done with an across doses non-inferiority test with a 4-fold margin (2 log 2) (Tobit model with Bonferroni correction for multiple comparisons). Dots represents estimated mean difference and horizontal whiskers represents confidence intervals. Dotted line at −2 indicates the pre-set non-inferiority margin. Dotted line at 0 indicates estimated mean of benchmark (preF without fusion inhibitor).

FIG. 16. RSV neutralization at day 42 in differentiated human airway cell cultures (hAEC). Mice were immunized at day 0 and 28 and serum was collected at day 42. VNA titers were measured in primary human airway cells differentiated at an air-liquid interface using an RSV-A2 strain encoding a GFP reporter. The entire transwell insert was imaged 4 days post-infection using a Cytation 1 automated microscope, infected cells are depicted in gray. Representative inserts of three biological replicates are shown.

### DETAILED DESCRIPTION OF THE INVENTION

Many of the circulating human pathogenic viruses are enveloped with a lipid bilayer and infect their target cells by inducing the fusion of the viral envelope with the target cell membrane. The viral fusion protein is the key factor that induces the membrane fusion reaction that allows viral entry. To deliver their RNA genome into host cells, these enveloped viruses have evolved a membrane fusion mechanism that in some viruses includes two surface glycoproteins: a receptor binding protein (e.g. the RSV G protein) and a fusion protein (e.g. the RSV F protein). A number of enveloped viruses, however, such as Ebola and HIV, display only a single protein on the particle surface, which necessarily mediates both the attachment to the cell surface as well as induces the subsequent membrane fusion reaction.

There are currently three classes of structurally distinct fusion proteins that have been characterized, termed class I, typified by influenza HA; class II, illustrated by the flavivirus envelope protein E; and class III, typified by the rhabdovirus glycoprotein G.

Fusion of the viral and host cell membranes is crucial in the life cycle of viruses using a class I fusion protein. The group of enveloped viruses carrying class I fusion proteins includes respiratory viruses such as the influenza viruses (four genera in the Orthomyxovirus family: influenza A, B, C and D), the respiratory syncytial virus (RSV, Pneumoviridae family) and the related measles, mumps and parainfluenza viruses in the Paramyxoviridae family, which also includes the recently emerged zoonotic Hendra and Nipah encephalitis viruses that cause serious disease in humans. Other respiratory virus members of the Class I group include the coronaviruses (CoVs) (Coronaviridae family) responsible for seasonal respiratory infections (NL73 CoV and HKU1 CoV, for instance), as well as the zoonotic severe acquired respiratory syndrome (SARS CoV) and Middle-Eastern respiratory syndrome coronaviruses (MERS CoV). The Retroviridae family, exemplified by HIV and the human T cell leukemia viruses (HTLVs), represent another important subset of class I viruses. Last but not least, several important hemorrhagic fever agents have class I fusion proteins, the most notable ones being Lassa virus together with other members of the Arenaviridae family, and Ebola virus and relatives in the Filoviridae family.

Although efficient vaccines against some of these viruses exist, for the majority there is no prophylactic or therapeutic treatment. The known vaccines typically work by eliciting antibodies that block entry of the pathogen into cells, which in the case of enveloped viruses typically involves antibody binding to the viral envelope attachment or fusion proteins.

As explained above, the immunogenic activity of fusion protein-based vaccines depends to a large extent on the structural integrity of the fusion protein antigens, especially in relation to conformational epitopes (where antibodies are required to bind disparate regions of the polypeptide chain brought together by native folding). Irreversible conformational changes and aggregation thus can lead to a reduced efficacy of vaccines. It is therefore very important that the structural characteristics of the fusion protein, in particular the secondary, tertiary and quaternary structure of the fusion protein in the vaccine are retained during production, transport and storage of the vaccine compositions.

It is generally known that class I fusion proteins typically are labile (metastable) proteins. For example, RSV F can adopt multiple conformations. Thus, on the viral surface, RSV F exists in a metastable pre-fusion conformation that, during the infection process, rearranges to a more stable post-fusion form to enable virus entry into the host cell. It has been shown that the majority of the neutralizing antibodies induced by natural RSV infection are directed towards epitopes specific for the pre-fusion conformation. Therefore, for development of efficacious vaccines based on RSV fusion protein it is desirable that this meta-stable fusion protein is stably maintained in the pre-fusion conformation.

Metastable class I fusion proteins that have been stabilized in the pre-fusion conformation with e.g. trimerization domains and stabilizing amino acid substitutions and that are stable for prolonged times at 2-8 degrees, upon agitation or multiple freeze/thaw cycles, however, can still form aggregates after more harsh conditions like a very slow freeze process of 24 hours, or may be unstable to heat, or long-term storage at 4° C., and thus can lose their potency upon storage. Improvement of the stability of such proteins can solve cold-chain problems that for example may be faced in remote and poorer areas and can prolong the shelf life of the vaccine.

The present invention provides vaccine compositions comprising an immunologically effective amount of a viral fusion protein antigen and a stabilizing amount of an antiviral compound, which compositions have an improved stability. In certain embodiments, the fusion protein is a metastable fusion protein. A metastable fusion protein is stable in the pre-fusion conformation but the stability is not sufficient to remain in the pre-fusion conformation. A particular trigger (receptor binding, drop in pH, higher temperature) may push the protein to the non pre-fusion conformation. As explained above, for their function, fusion proteins need to be unstable or metastable. For vaccine purposes, however, it is important to stabilize them in the pre-fusion conformation. In case of instability and even metastability, the pre-fusion conformation may not ‘survive’ manufacturing and storage until the use of the vaccine.

In a preferred embodiment, the fusion protein is a (metastable) trimeric class I fusion protein.

As used herein, “an immunologically effective amount” means an amount of an antigen that is sufficient to induce a desired immune effect or immune response in a subject in need thereof. In certain embodiments, an immunologically effective amount means an amount that is sufficient to induce immunity in a subject in need thereof, e.g., provide a protective effect against a viral infection. In certain embodiments, an immunologically effective amount means an amount that is sufficient to enhance an immune response in a subject in need thereof. For example, when used in combination with one or more other components or immunogenic compositions capable of effecting an immune response, such as in a prime-boost regimen, an immunologically effective amount can be an amount sufficient to enhance the immune response induced by the one or more other components or immunogenic compositions. An immunologically effective amount can vary depending upon a variety of factors, such as the physical condition of the subject, age, weight, health, the particular application, e.g., whether inducing immune response or providing protective immunity, and the viral infection for which immunity is desired. An effective amount can readily be determined by one of ordinary skill in the art.

As used herein an improved (or increased) stability means an improved (or increased) stability as compared to compositions comprising the immunologically effective amount of said fusion protein antigen without the antiviral compound. The terms improved and increased are used interchangeably in this respect. The improved (or increased) stability can comprise an improved (or increased) thermostability, an improved (or increased) stability against agitation stress, an improved (or increased) stability against thermal stress (e.g. freeze-thaw stress), and/or an improved (or increased) stability against stress by pH fluctuations. As used herein thermostability refers to the quality of the protein antigen to resist irreversible change in its chemical or physical structure at a high relative temperature.

According to the invention it has been shown that viral fusion protein antigens, in particular (metastable) class I fusion proteins, can be stabilized by adding a stabilizing amount of an antiviral compound, which is known to bind to and/or to interfere with the function of said viral protein. The compositions of the invention have an increased stability. The invention is particularly applicable to vaccine compositions in which the retention of structural characteristics of a protein, in particular the secondary, tertiary and quaternary structure, are of importance. The application of the invention significantly reduces the probability of irreversible conformational change and irreversible aggregation of the protein antigen and consequent loss of the capacity to induce an immune response against the native protein. The invention is applicable to stabilization of a protein antigen throughout its complete product life, including, but not limited to, isolation or expression of the protein antigen, purification thereof, manufacture of the vaccine product and transport and storage thereof.

Antiviral compounds are a category of antimicrobial drugs used specially for treating viral infections by inhibiting the development of the viral pathogen inside the host cell. According to the invention, the antiviral compound may be any antiviral compound which is known to bind to and/or to interfere with the entry of a virus into a target cell. According to the invention, the antiviral compound typically is a small molecule compound, i.e. a low molecular weight (<900 daltons) organic compound. Many known drugs are small molecules. In certain embodiments, the antiviral compound is a fusion inhibitor and/or a viral entry inhibitor. Small molecule fusion inhibitors and/or viral entry inhibitors are known and are typically used in (experimental) treatment of viral infections caused by viruses such as RSV, HW or influenza.

According to the invention a ‘stabilizing amount” of said fusion and/or viral entry inhibitor typically is an amount which is sufficient for stabilizing the protein antigen, but which is below the therapeutically effective amount of said compounds.

In certain preferred embodiments, the stabilizing amount of the antiviral compound is a sub-therapeutically effective amount of said antiviral compound. Thus, when administered as a vaccine, the antiviral compound will not exert any therapeutic (antiviral) effect.

In certain embodiments, the stabilizing amount of the antiviral compound is at least 100×, preferably at least 1000×, more preferably at least 10,000× or at least 100,000× lower, for example 500,000 times, lower than the therapeutically effective amount of said antiviral compound.

In certain embodiments, the trimeric fusion protein and said antiviral compound are present in a trimer: compound ratio ranging between and including 1:1 and 1:300, preferably between 1:1 and 300,000. such as but not limited to a ratio of 1:3, 1:9, 1:10, 1:27, 1:30, 1:50, 1:150, 1:100, or 1:300. According to the invention, a trimer:compound ratio of, for example, 1:3 means 1 molar equivalent of fusion protein trimer, for example RSV F protein trimer, and 3 molar equivalents of inhibitor compound.

In certain embodiments, the fusion protein is an RSV fusion (F) protein, preferably a pre-fusion RSV F protein, i.e. an F protein that is stabilized in the pre-fusion conformation, for example by stabilizing mutations and/or addition of a heterologous trimerization domain.

The fusion (F) protein of RSV is typically expressed as a single precursor of 574 amino acids with several sites of N-linked glycosylation. This precursor molecule F0 oligomerises in the endoplasmic reticulum and is proteolytically processed at two sites in each monomer, resulting in a trimer of two disulphide-linked fragments: F2 (the smaller N-terminal fragment) and F1. The protein is anchored to the virion membrane through a hydrophobic peptide in the C-terminal region of F1 and is believed to adopt a metastable pre-fusion conformation until it is triggered. Triggering can happen even without binding to a target membrane and/or receptor. The RSV F protein contains two heptad repeat domains, HR1 (also known as HRA) and HR2 (also known as HRB). During fusion, a folding intermediate of the fusion protein is formed which contains a coiled-coil structure of three HR1 domains. This trimeric coiled-coil structure irreversibly refolds into a ‘six-helix bundle’ (6HB)-complex with three HR2 domains, juxtaposing the viral and cellular membrane.

Pre-fusion RSV F proteins have been described previously. The present invention is applicable to several pre-fusion F proteins, including, but not limited to the pre-fusion RSV F proteins as described in WO2014/174018, WO2014/202570, WO2017/005844, WO2017/174568 and WO2017/207480. A preferred RSV F protein is the pre-fusion F protein of SEQ ID NO: 1 which will be processed at 2 furin cleavage sites, cleaving out the p27 region resulting in a processed F protein composed of F2 and F1 held together by disulphide bridges, yielding a processed protein with the amino acid sequence of SEQ ID NO: 10.

According to the invention, the antiviral compound may be any small molecule compound that binds to and/or interferes with the fusion of an RSV virus to the target cell. The skilled person will be able to identify suitable fusion or entry inhibitors. For example, the antiviral compound may be an RSV entry and/or fusion inhibitor, identified as described in WO2009/106580 and analogues thereof.

In certain embodiments, the antiviral compound is an RSV F entry or fusion inhibitor selected from the group consisting of compound I-XVI in Table 1 and suitable analogues thereof.

In a preferred embodiment, the antiviral compound is 3-[[5-bromo-1-(3-methylsulfonylpropyl)benzimidazol-2-yl]methyl]-1-cyclopropyl-imidazo[4,5-c]pyridin-2-one (Compound III).

TABLE 1
RSV compound overview (*IUPAC names are automatically generated
(workflow uses Accelrys Direct, Revision 8.0 SP1 (Microsoft
Windows 64-bit Oracle11) (8.0.100.4), OpenEye: 1.2.0))

| COMPOUND | IUPAC NAME* |
|---|---|
| I | 2-[[6-[[2-(3-hydroxypropyl)-5-methyl-anilino]methyl]-2-(3- |
| | morpholinopropylamino)benzimidazol-1-yl]methyl]-6-methyl-pyridin-3-ol |
| II | 3-[[5-chloro-1-(3-methylsulfonylpropyl)benzimidazol-2-yl]methyl]-1- |
| | cyclopropyl-imidazo[4,5-c]pyridin-2-one |
| III | 3-[[5-bromo-1-(3-methylsulfonylpropyl)benzimidazol-2-yl]methyl]-1- |
| | cyclopropyl-imidazo[4,5-c]pyridin-2-one |
| IV | 3-[[5-chloro-1-(3-methylsulfonylpropyl)indol-2-yl]methyl]-1-(2,2,2- |
| | trifluoroethyl)imidazo[4,5-c]pyridin-2-one |
| V | [1′-[[5-chloro-1-(3-cyanopropyl)benzimidazol-2-yl]methyl]-2′-oxo- |
| | spiro[cyclobutane-3,3′-indoline]-1-yl] N-cyclopropylcarbamate |
| VI | 3-[[5-bromo-1-(4-fluorobutyl)benzimidazol-2-yl]methyl]-1-cyclopropyl- |
| | imidazo[4,5-c]pyridin-2-one |
| VII | 1′-[[5-chloro-1-(3-methylsulfonylpropyl)indol-2-yl]methyl]-1-methylsulfonyl- |
| | spiro[piperidine-4,3′-pyrrolo[2,3-c]pyridine]-2′-one |
| VIII | 4-[5-chloro-2-[(1-isopropenyl-2-oxo-imidazo[4,5-c]pyridin-3- |
| | yl)methyl]benzimidazol-1-yl]butanenitrile |
| IX | 3-[[5-chloro-1-(4-fluorobutyl)benzimidazol-2-yl]methyl]-1-(2,2- |
| | difluoroethyl)imidazo[4,5-c]pyridin-2-one |
| X | 1′-[[5-chloro-1-(3-methylsulfonylpropyl)benzimidazol-2- |
| | yl]methyl]spiro[cyclopentane-1,3′-pyrrolo[2,3-c]pyridine]-2′-one |
| XI | 3-[[5-chloro-1-(4,4,4-trifluorobutyl)benzimidazol-2-yl]methyl]-1-(2,2,2- |
| | trifluoroethyl)imidazo[4,5-c]pyridin-2-one |
| XII | 1′-[[5-chloro-1-(3-methylsulfonylpropyl)benzimidazol-2-yl]methyl]-1-(3- |
| | pyridyl)spiro[azetidine-3,3′-indoline]-2′-one |
| XIII | 1-[[5-chloro-1-(4,4,4-trifluorobutyl)benzimidazol-2- |
| | yl]methyl]spiro[pyrrolo[2,3-c]pyridine-3,4′-tetrahydropyran]-2-one |
| XIV | N-[1′-[[5-chloro-1-(3-cyanopropyl)indol-2-yl]methyl]-2′-oxo- |
| | spiro[piperidine-4,3′-pyrrolo[2,3-c]pyridine]-1-yl]sulfonylacetamide |
| XV | 4-[5-chloro-2-[[1-(1-hydroxycyclopropanecarbonyl)-2′-oxo- |
| | spiro[azetidine-3,3′-indoline]-1′-yl]methyl]indol-1-yl]butanenitrile |
| XVI | [1′-[[5-chloro-1-(3-methylsulfonylpropyl)benzimidazol-2-yl]methyl]-2′- |
| | oxo-spiro[cyclobutane-3,3′-indoline]-1-yl] morpholine-4-carboxylate |

In other embodiments, the class I fusion protein is an HIV envelope (env) protein, preferably a pre-fusion HIV env protein. The envelope (Env) protein of HIV is expressed on the envelope of an HIV virion and enables an HIV to target and attach to the plasma membrane of HIV target cells and fuse the viral and target cell membranes

Pre-fusion HIV env proteins have been described previously. The invention is not limited to a particular pre-fusion HIV env protein. Suitable HIV env proteins are for example described by Rutten et al. (Cell Reports 23: 584-595 (2018)). A preferred HIV env is the pre-fusion HIV env protein of SEQ ID NO: 2.

As indicated above, according to the invention, the antiviral compound may be any small molecule compound that binds to and/or interferes with the entry of the HIV virus in the target cell. The skilled person will be able to identify suitable fusion or entry inhibitors. In certain embodiments, the antiviral compound is an HIV fusion and/or entry inhibitor, such as, but not limited to an HIV fusion or entry inhibitor selected from the group consisting of compounds XVII-XXIII in Table 2 and suitable analogues thereof.

TABLE 2
HIV compound overview (*IUPAC names are automatically generated
(workflow uses Accelrys Direct, Revision 8.0 SP1 (Microsoft
Windows 64-bit Oracle11) (8.0.100.4), OpenEye: 1.2.0))

| Compound | IUPAC name* |
|---|---|
| XVII | 1-[(2R)-4-benzoyl-2-methyl-piperazin-1-yl]-2-(4-methoxy-1H-pyrrolo[2,3- |
| | b]pyridin-3-yl)ethane-1,2-dione |
| XVIII | 1-[(2R)-4-benzoyl-2-methyl-piperazin-1-yl]-2-(1H-pyrrolo[2,3-b]pyridin-3- |
| | yl)ethane-1,2-dione |
| XIX | 1-(4-benzoylpiperazin-1-yl)-2-(4-methoxy-1H-pyrrolo[2,3-b]pyridin-3-yl)ethane- |
| | 1,2-dione |
| XX | 1-(4-methoxy-1H-pyrrolo[2,3-b]pyridin-3-yl)-2-(3-phenyl-6,8-dihydro-5H- |
| | [1,2,4]triazolo[4,3-a]pyrazin-7-yl)ethane-1,2-dione |
| XXI | 1-(4-methoxy-1H-pyrrolo[2,3-b]pyridin-3-yl)-2-(8-methyl-3-phenyl-6,8-dihydro- |
| | 5H-[1,2,4]triazolo[4,3-a]pyrazin-7-yl)ethane-1,2-dione |
| XXII | 1-(4-methoxy-1H-pyrrolo[2,3-b]pyridin-3-yl)-2-[3-(3-thienyl)-6,8-dihydro-5H- |
| | [1,2,4]triazolo[4,3-a]pyrazin-7-yl]ethane-1,2-dione |
| XXIII | 1-(4-methoxy-1H-pyrrolo[2,3-b]pyridin-3-yl)-2-[3-(3-thienyl)-6,8-dihydro-5H- |
| | [1,2,4]triazolo[4,3-a]pyrazin-7-yl]ethane-1,2-dione |

In further embodiments, the class I fusion protein is an influenza hemagglutinin (HA) protein, preferably a pre-fusion HA protein. Hemagglutinin (HA) is the major envelope glycoprotein from influenza viruses. HA has two main functions during the entry process. First, hemagglutinin mediates attachment of the virus to the surface of target cells through interactions with sialic acid receptors. Second, after endocytosis of the virus, HA subsequently triggers the fusion of the viral and endosomal membranes to release its genome into the cytoplasm of the target cell. HA comprises a large ectodomain of ˜500 amino acids that is cleaved by host-derived enzymes to generate 2 polypeptides (HA1 and HA2) that remain linked by a disulfide bond. The majority of the N-terminal fragment (the HA1 domain, 320-330 amino acids) forms a membrane-distal globular “head domain” that contains the receptor-binding site and most determinants recognized by virus-neutralizing antibodies. The smaller C-terminal portion (HA2 domain, ˜180 amino acids) forms a stem-like structure that anchors the globular domain to the cellular or viral membrane.

In certain embodiments, the class I fusion protein is an influenza hemagglutinin (HA) A or B protein, preferably a pre-fusion HA A or B protein.

According to the invention, the antiviral compound may be any small molecule compound that binds to and/or interferes with the entry of the influenza virus in the target cell. The skilled person will be able to identify suitable fusion or entry inhibitors. In certain embodiments the antiviral compound is an influenza fusion and/or entry inhibitor. In certain embodiments, the antiviral compound is selected from the group consisting of compounds XXIV-XXVI in Table 3, and suitable analogues thereof, for influenza A HA, or compound XVII, or suitable analogues thereof. for influenza B HA.

Table 3. Influenza compound overview (*IUPAC names are automatically generated (workflow uses Accelrys Direct, Revision 8.0 SP1 (Microsoft Windows 64-bit Oracle11) (8.0.100.4), OpenEye:1.2.0))
Compound IUPAC name*
XXIV ethyl 6-bromo-4-(dimethylaminomethyl)-5-hydroxy-1-methyl-2-(p-tolylsulfanylmethyl)indole-3-carboxylate (Arbidol)
XXV 2-tert-butylbenzene-1,4-diol (TBHQ)
XXVI [4-[(R)-(2-methyltetrazol-5-yl)-phenyl-methyl]piperazin-1-yl]-[4-[5-(trifluoromethoxy)-1,3-benzoxazol-2-yl]-2-pyridyl]methanone
XXVII 1-benzyl-N,4-diphenyl-piperidin-4-amine

In certain embodiments, the vaccine composition is a liquid composition. Liquid compositions that are stable under frozen conditions (−80° C.) typically require specialized shipment and expensive storage facilities, making a reliable cold chain almost impossible, especially at the periphery of the distribution network. A preferred vaccine composition is therefore a liquid composition with an increased stability, such as an increased thermostability at a temperature range between 2-8° C., but also at higher temperatures, such as at room temperature or even higher (e.g. 37° C.), and that also remains stable even after a very slow freeze-thaw process of 24 hours. Such a composition can be stored in a regular fridge and can be administered quickly and easily. In addition, storage at refrigerated but not frozen conditions ensure that the vaccine compositions can be used more easily in e.g. resource-limited settings e.g. where no freezing capacity is available. Moreover, the observed maintained stability at low or elevated temperatures (e.g. 25° C., 37° C. and even 47° C.) of the compositions of the invention indicates that inadvertent temperature excursions, e.g. non-intended freezing or when temporarily the composition is exposed to room temperature even in warm climates, should not immediately be detrimental to the vaccine composition of the invention.

In certain embodiments, the vaccine composition has an improved stability upon storage at a temperature ranging between room temperature and 47° C. for at least 6 weeks. Thus, according to the invention the vaccine composition has an improved stability upon storage at increased temperatures, such as for example at room temperature, or even higher temperatures up to 47° C.

In certain embodiments, the vaccine composition has an improved stability after slow-freezing to a temperature between −20 and −80° C. and subsequent thawing of said composition.

In certain embodiments, the vaccine composition according to the invention has an improved stability upon ultrafiltration/diafiltration.

A vaccine composition according to the invention either refers to a drug substance or drug product. The drug product typically is a finished dosage form, e.g., tablet, capsule, or solution (formulation), that contains a drug substance, generally, but not necessarily, in association with one or more other ingredients. The drug substance is an active ingredient that is intended to provide pharmacological activity or other direct effect in the diagnosis, cure, mitigation, treatment, or prevention of disease or to affect the structure or any function of the human body. The vaccine compositions according to the invention as described herein, can be formulated in any matter suitable for administration to a human subject to facilitate administration and improve efficacy, including, but not limited to, oral (enteral) administration and parenteral injections. The parenteral injections for instance can include subcutaneous injection, intramuscular injection, or intradermal injection. Immunogenic compositions of the invention can also be formulated for other routes of administration, e.g. transmucosal, rectal, sublingual administration, oral, or intranasal. Preferably, an immunogenic composition is formulated for intramuscular injection.

As indicated above, the vaccine composition of the invention may further comprise one or more pharmaceutically acceptable excipients. By “pharmaceutically acceptable excipient” is meant any inert substance that is combined with an active molecule such as an antigen for preparing an agreeable or convenient dosage form. The “pharmaceutically acceptable excipient” is an excipient that is non-toxic to recipients at the dosages and concentrations employed and is compatible with other ingredients of the composition comprising antigen. Examples of excipients are cryoprotectants, non-ionic detergents, buffers, and salts.

The vaccine compositions may further comprise an adjuvant. The term “adjuvant” is defined as one or more substances that cause stimulation of the immune system or enhance an immune response.

The present invention further provides methods for preparing a vaccine composition comprising a protein antigen, said method comprising admixing an immunologically effective amount of said fusion protein antigen as described herein with a stabilizing amount of an antiviral compound as described herein.

In certain embodiments, the composition is stable upon storage at an increased temperature, such as for example at room temperature, or even higher temperatures up to 47° C. for 6 weeks. According to the invention “stable” means that aggregation of the protein is absent or reduced as compared to the same composition without inhibitor compound.

In certain embodiments, the vaccine composition has an improved stability after slow-freezing to a temperature between −20 and −80° C. and subsequent thawing of said composition.

The invention further provides methods for reducing aggregation of viral fusion proteins in a vaccine composition, comprising admixing an immunologically effective amount of a fusion protein antigen as described herein with a stabilizing amount of an antiviral compound as described herein.

The invention in particular provides methods for reducing aggregation of viral fusion proteins in a vaccine composition after freeze-thawing of said vaccine composition, comprising preparing a vaccine composition by admixing an immunologically effective amount of a fusion protein antigen as described herein with a stabilizing amount of an antiviral compound as described herein.

The invention also provides methods of preserving a vaccine comprising a fusion protein antigen, which method comprises preparing a vaccine composition as described herein.

In certain embodiments, said methods further comprise storing said composition at a temperature ranging between 2-8° C. for at least 24 months.

Furthermore, the invention provides methods for stably maintaining a liquid vaccine composition comprising a protein antigen, the method comprising storing a vaccine composition as described herein at a temperature of 2-8° C. for at least 24 months.

The invention also provides a method of producing a fusion protein immunogen, comprising producing the fusion protein immunogen in the presence of a stabilizing amount of an antiviral compound. According to the invention it has been shown that by producing (e.g. expressing) the fusion protein in the presence of an antiviral compound, expression levels and yields are increased.

The detailed examples which follow are intended to contribute to a better understanding of the present invention. However, the invention is not limited by the examples. Other embodiments of the invention will be apparent to those skilled in the art from consideration of the specification and practice of the invention disclosed herein.

### EXAMPLES

### Example 1: RSV Prefusion F Protein

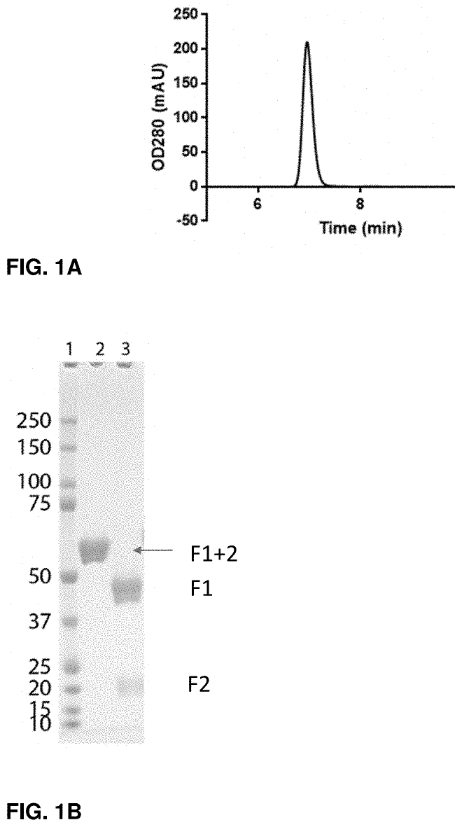

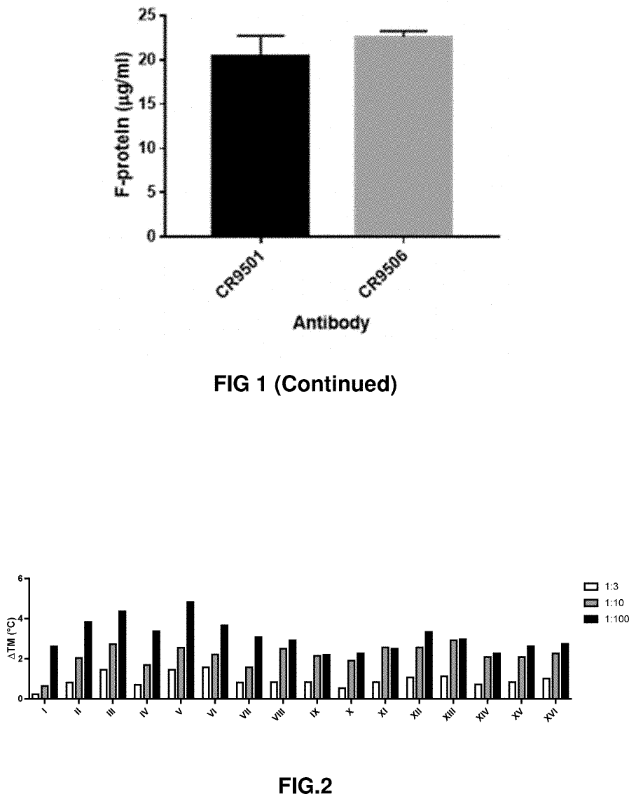

Recombinant soluble RSV F stabilized in the pre-fusion conformation (SEQ ID NO: 10) was purified and analyzed on analytical SEC (FIG. 1A) and SDS-PAGE (FIG. 1B). Proteins were visualized on the gel upon staining with Coomassie Brilliant Blue. Main bands on the SDS-PAGE are corresponding to F1 and F2 domains of RSV F protein in reduced samples; in non-reduces samples the main band corresponds to the size of F1+F2 domains linked together with disulfide bonds. Pre-fusion and post-fusion binding antibodies CR9506 (comprising the heavy and light chain variable region of SEQ ID NO: 8 and SEQ ID NO: 9, respectively) was used to measure RSV F protein (FIG. 1C). The pre-fusion conformation of the purified protein was confirmed by binding to CR9501 (comprising the binding regions of the antibody 58C5 as described in WO2011/020079) (FIG. 1C).

In a separate experiment the stabilized pre-fusion RSV F protein remained stable in the pre-fusion conformation at 2-8° C. for more than 24 months (data not shown).

### Example 2 Thermo-Stability of RSV Prelusion F with and without Inhibitor

Thermo-stability of the RSV pre-fusion F protein of SEQ ID NO: 10 was determined by Differential Scanning Fluorimetry (DSF) by monitoring the fluorescent emission of Sypro Orange Dye (ThermoFisher Scientific) in a 96 well optical qPCR plate. 15111 of a 66.67 μg/ml polypeptide solution was used per well, with and without inhibitors I-XVI (Table 1) as shown in FIG. 2. Inhibitors were diluted in DMSO prior to the experiment. Same amount of DMSO was added to pre-fusion F protein without inhibitor. To each well, 5 μl of 20× Sypro orange solution was added. Upon gradual increase of the temperature, from 25° C. to 95° C. (0.015° C./s), the polypeptides unfold and the fluorescent dye binds to the exposed hydrophobic residues leading to a characteristic change in emission. The melting curves were measured using a ViiA7 real time PCR machine (Applied Biosystems). The 1st derivative of the fluorescent signal (a.u.) versus the temperature (° C.) of three individual samples (technical triplicate), as well as the averaged melting curve, were plotted with Graphpad Prism software (Dan Diego, CA, US). From the averaged melting curve, the Tm₅₀ was deducted (lowest point on the curve). The Tm₅₀ values represent the temperature at which 50% of the protein is unfolded and thus are a measure for the temperature stability of the polypeptides. The increase of the Tm₅₀ is reported as difference of the Tm₅₀ of pre-fusion F protein with stabilizing antiviral compound compared with the Tm₅₀ of pre-fusion F protein with only addition of DMSO (i.e. without antiviral compound).

As shown in FIG. 2, the addition of 16 different fusion inhibitors at a 1:3, 1:10 or a 1:100 trimer: inhibitor molar ratio increased the Tm₅₀ value, indicating an increased thermostability of the protein.

### Example 3: Antigenicity of RSV Prefusion F with Inhibitor

Binding of antibodies to the polypeptide (SEQ ID NO: 10) with and without addition of a stabilizing compound was measured by Enzyme-Linked Immuno Sorbent Assay (ELISA) (Table 4). First, 96 well half area HB plates (Perkin Elmer, cat #6002290) were coated with different antibodies (1 μg/mL) in Phosphate Buffer Saline (PBS), 50 μL/well and the plates were incubated overnight at 4° C. Pre-fusion specific antibodies used: CR9501, CR9502 (comprising the binding regions of the antibody 30D8 as described in WO2012/006596). Pre-fusion and post-fusion binding antibodies used: CR9506, CR9509 (comprising the binding regions of the antibody 17C9, as described in WO2012/006596). After incubation overnight, the plates were washed 3 times with 100 μL wash buffer (PBS+0.05% Tween20). To each well 100 μL blocking buffer was added (2% Bovine Serum Albumin (BSA), 0.05% Tween20 in PBS) and the plates were incubated for 1 hour at room temperature, shaking. Next, the plates were washed 3 times with 100 μL wash buffer (PBS+0.05% Tween20). For the sample preparation, the polypeptide samples with and without addition of stabilizing compound were first diluted to 4 μg/mL in assay buffer (1% BSA, 0.05% Tween20 in PBS). For the samples with stabilizing compound the compound was in a final concentration of 73.5 nM, 245 nM and 1125 nM. The 4 μg/mL samples (with and without compound) were diluted further 4-fold by adding 250 μL dilution to 750 μL assay buffer. The plates were incubated for 1 hour at room temperature, shaking. After incubation the plates were washed 3 times with 300 μL wash buffer. To each well 50 μL of the pre- and post-fusion binding antibody CR9506 with a horseradish peroxidase (HRP) label was added at a concentration of 0.05 μg/mL. The plates were incubated for 1 hour at room temperature, shaking. After incubation the plates were washed 3 times with 300 μL wash buffer and to each well 20 μL of the POD substrate was added. The plates were measured (EnSight Multimode Plate reader, HH34000000, reading Luminescence) between 5-15 minutes after addition of the substrate. The ELISA curve (protein dilution vs. RLU) were plotted in GraphPad Prism and GraphPad Prism was used to calculate the IC50 values (Table 4). The IC50 values of RSV prefusion F without and with compound are very comparable and show that the addition of fusion inhibitor does have no influence on the antigenicity of the prefusion F protein.

TABLE 4
IC50 values (nM) in ELISA for RSV prefusion
F with and without inhibitor III or VII

| | | preF + III | | | preF + VII | | | |
|---|---|---|---|---|---|---|---|---|
| Antibody | | Ratio trimer:compound | | | | | | preF + |
| (specificity) | PreF | 1:3 | 1:10 | 1:50 | 1:3 | 1:10 | 1:50 | DMSO |
| CR9501 (pre) | 0.57 | 0.65 | 0.66 | 0.71 | 0.71 | 0.65 | 0.72 | 0.63 |
| CR9502 (pre) | 0.14 | 0.16 | 0.17 | 0.16 | 0.14 | 0.15 | 0.16 | 0.14 |
| CR9506 (pan) | 0.28 | 0.30 | 0.29 | 0.31 | 0.32 | 0.29 | 0.31 | 0.29 |
| CR9509 (pan) | 0.09 | 0.10 | 0.10 | 0.11 | 0.09 | 0.09 | 0.10 | 0.10 |

### Example 4: Cryoprotective Effect of Small Molecule Inhibitor on RSV Prefusion F

The RSV pre-fusion F protein of SEQ ID NO: 10 was diluted to 0.3 mg/ml in phosphate buffer (Formulation 1) and 0.75 ml was filled in glass injection vials with rubber stopper and sealed with aluminum caps. Additionally, protein was diluted in the formulation buffers with inhibitor in a 1:3 trimer:compound ratio. One inhibitor binds one trimer so with a 1:3 ratio there is an excess of inhibitor. This resulted in a concentration of the inhibitor compound of 5.2×10⁻⁶ M. Vials were subjected to slow-freezing stress using an environmental simulation chamber (Binder, model MKT 115). In 24 hours, samples were cooled down from RT to −70° C. Samples were thawed to RT and analyzed by analytical Size Exclusion Chromatography (SEC) to measure loss of RSV F trimer.

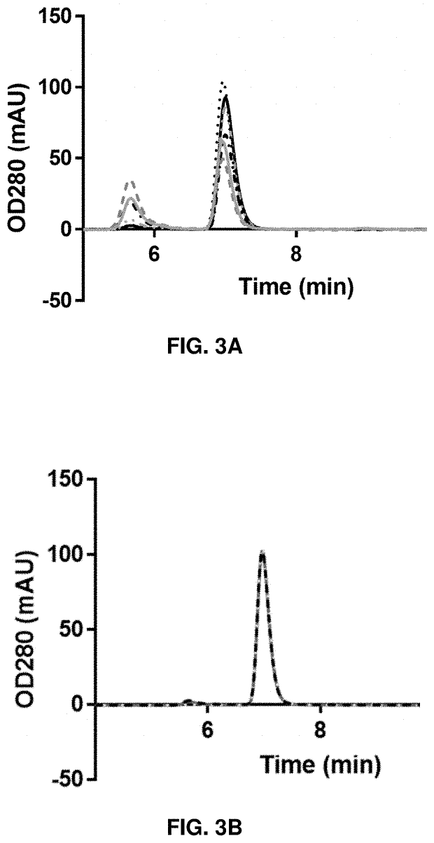

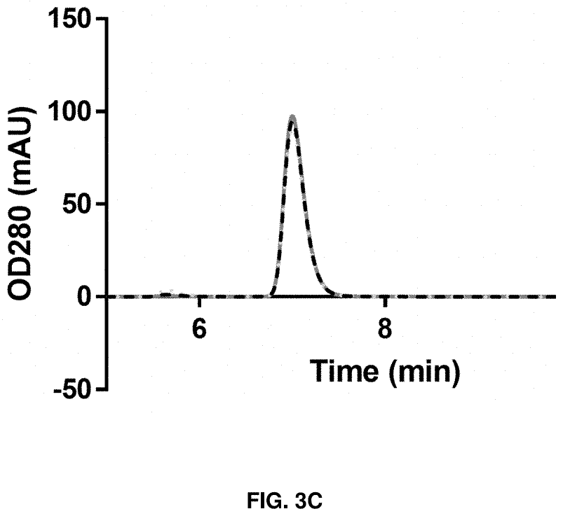

FIG. 3A shows that preF protein without stabilizing compound has a tendency to aggregate after the slow freeze/thaw process and that 20+/−17% of the trimer signal is lost. However, when compound II (FIG. 3B) or III (FIG. 3C) was added to the composition, aggregation was strongly reduced and only 2.4+/−1.9% or 2.0+/−2.0% of trimer signal was lost respectively, indicating a strong cryoprotective effect of both stabilizing compounds.

### Example 5. Cryoprotective Effect of Small Molecule Inhibitor on RSV Prefusion F in Different Formulation Buffers

The RSV pre-fusion F (preF) protein of SEQ ID NO: 10 was dialyzed to different formulation buffers (Table 5) and each formulation was diluted to 0.3 mg/ml. 0.75 ml of each formulation was filled in glass injection vials with rubber stopper and sealed with aluminum caps. Additionally, protein was diluted in the formulation buffers and inhibitor was added in a 1:1, 1:3, 1:9, 1:27 and 1:50 trimer: compound ratio. Final compound concentrations were 1.7×10⁻⁶ M, 5.2×10⁻⁶ M, 1.6×10⁻⁵M, 4.6×10⁻⁵, and 8.6×10⁻⁵ M, respectively. Vials were slowly frozen to −70° C. in 24 hours. Samples were subsequently thawed to RT and analyzed by analytical Size Exclusion Chromatography (SEC) to measure loss of RSV F trimer compared the sample that was kept at 4° C.

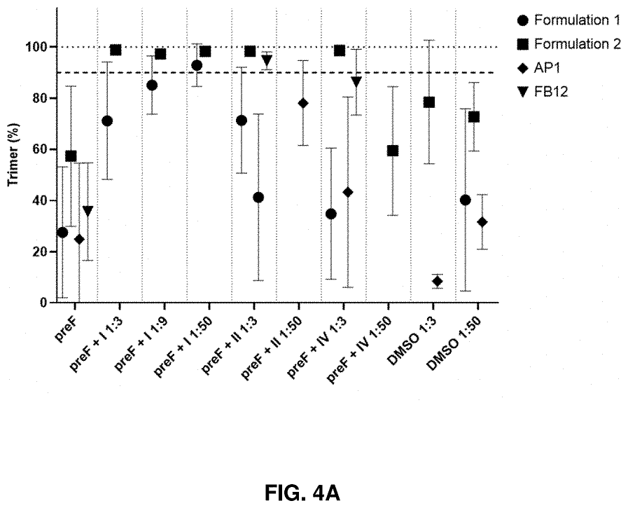

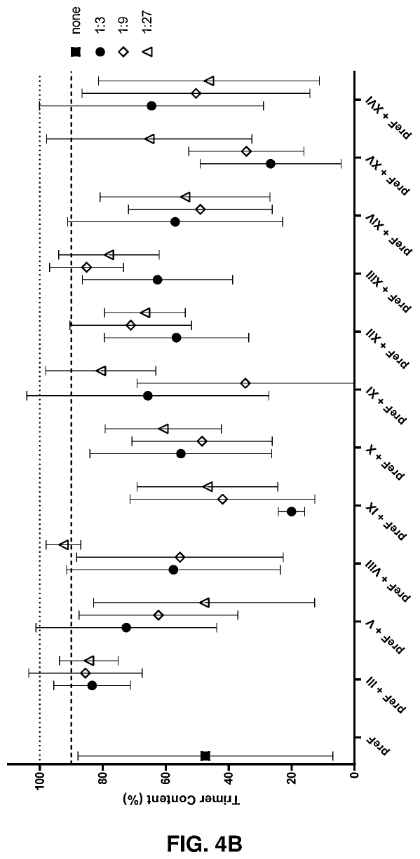

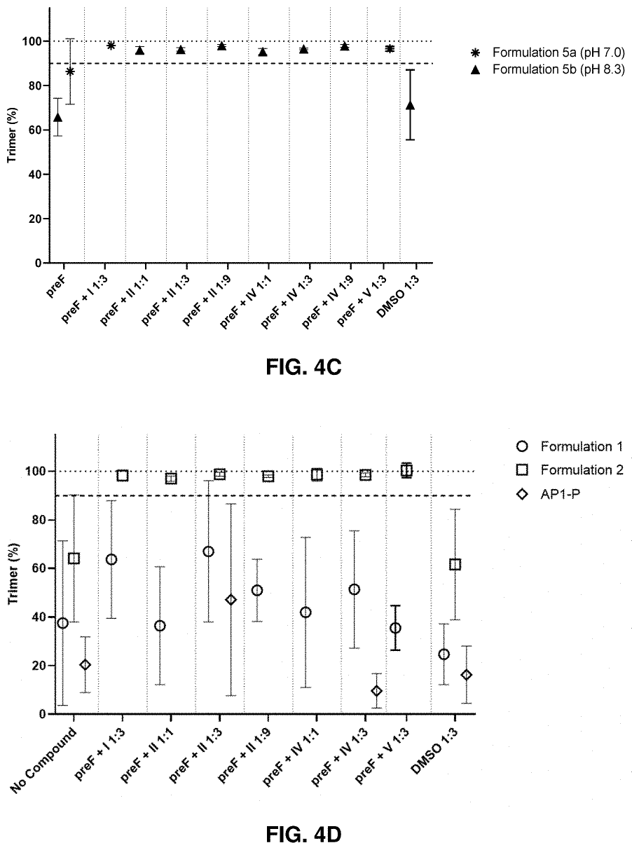

FIG. 4A shows that without stabilizing compound or with the addition of the compound diluent (DMSO), the preF protein has a tendency to aggregate in all tested formulations. Depending on the formulation the trimer loss ranges from 43% (formulation 2) to more than 60% for (formulation 1, AP1 and FB12). However, when compound I, II or IV were added to the composition a higher trimer % was observed for all formulation buffers, indicating a strong cryoprotective effect of all three compounds.

Additional compounds were evaluated in formulation 1 (FIG. 4B) and depending on the compound and concentration a loss in trimer after slow freeze could be prevented. For formulation 5b (pH 8.3) and 5a (pH 7.0) no aggregations were observed when compound I, II or IV (Table 1) were added (FIG. 4C).

Testing the different formulations in the absence of Polysorbate-20 is summarized in FIG. 4D. Also, in this case, preF protein without stabilizing compound tended to aggregate. The addition of fusion inhibitor to a formulation 2 without polysorbate resulted in a high trimer recovery above 90%. For formulation 1 without polysorbate a dose dependent improvement in trimer percentage was observed. The results display a strong cryoprotective effect of the compounds in formulations without Polysorbate-20.

Number of replicates tested: A: PS4P preF n=20; TS5P2 preF n=20; AP1 preF n=10; FB12 preF n=5; PS4P/TS5P2 preF+I 1:3/1:9/1:50 n=5; PS4P preF+II 1:3 n=15; TS5P2/AP1 preF+II 1:3/1:50 n=10; FB12 preF+II 1:3 n=5; PS4P/AP1 preF+IV 1:3 n=10; TS5P2/FB12+preF IV 1:3 n=5; AP1+preF IV 1:50 n=5; TS5P2/AP1 preF+DMSO 1:3 n=5; PS4P/TS5P2/AP1 preF+DMSO 1:50 n=5. B: PS4P: preF n=10; preF+III/V/VIII/XII/XIII 1:3/1:9 n=10; preF+III/V/VIII/XII/XIII 1.27 n=5; preF+IX/X/XI/XIV/XV/XVI n=5. C: n=5. D: PS4 preF n=15; TS5 preF n=10; AP1-P preF n=5; PS4/TS5P preF+II 1:1 n=5; PS4 preF+II 1:3 n=10; TS5P/AP1-1 preF+II 1:3 n=5; PS4/TS5P preF+II 1:9 n=5; PS4/TS5P preF+IV 1:1 n=5; PS4 preF+IV 1:3 n=10; TS5P/AP1-1 preF+IV 1:3 n=5; PS4/TS5P preF+V 1:3 n=5; PS4/TS5P preF+I 1:3 n=5; PS4/TS5P/AP1-1 preF+DMSO 1:3 n=5.)

TABLE 5
Composition of formulations AP1 and FB12

| Name | Buffer | pH | Components | | | |
|---|---|---|---|---|---|---|
| AP1 | 15 mM | 6.2 | 75 mM | 5% | 0.03% | 0.4% EtOH |
| | Citrate | | NaCl | HBCD | Polysorbate- | |
| | | | | | 80 | |
| FB12 | 20 mM | 6.5 | 75 mM | 5% | 0.02% | 0.4% EtOH, |
| | Histidine | | NaCl | Sucrose | Polysorbate- | 0.1 mM |
| | | | | | 20 | EDTA |
| AP1 -P | 15 mM | | 75 mM | 5% | | 0.4% EtOH |
| | Citrate | | NaCl | HBCD | | |

### Example 6: Stabilizing Effect of Small Molecule Inhibitor on RSV Prefusion F in Different Formulation Buffers at Different Temperatures

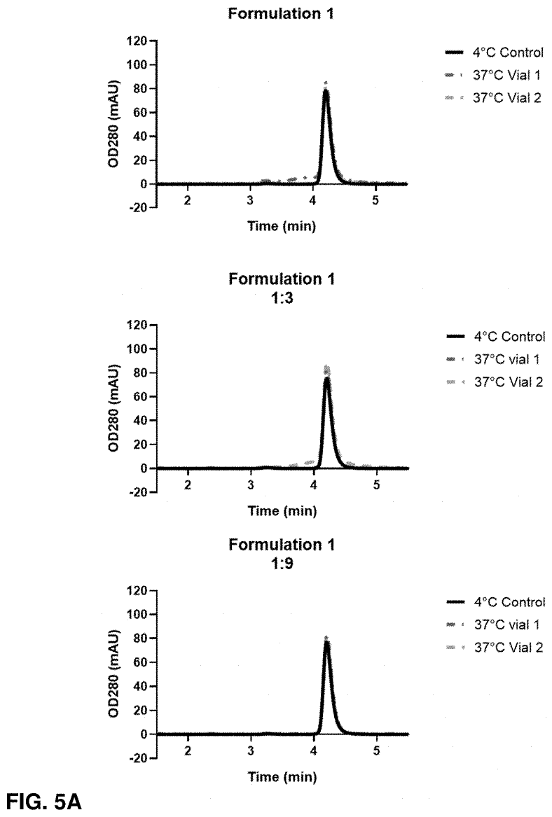

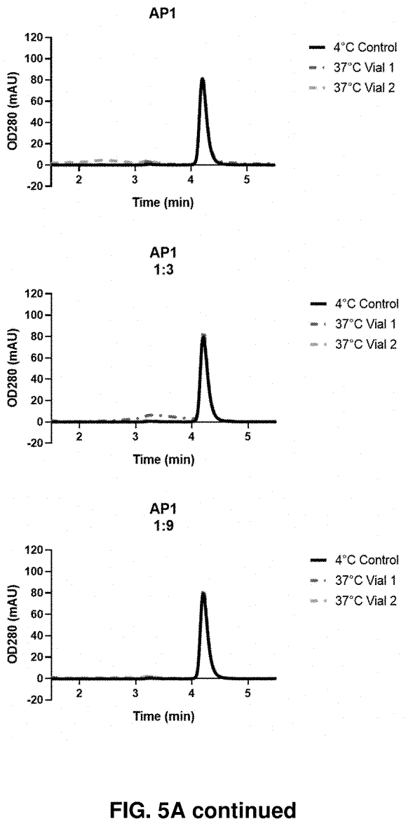

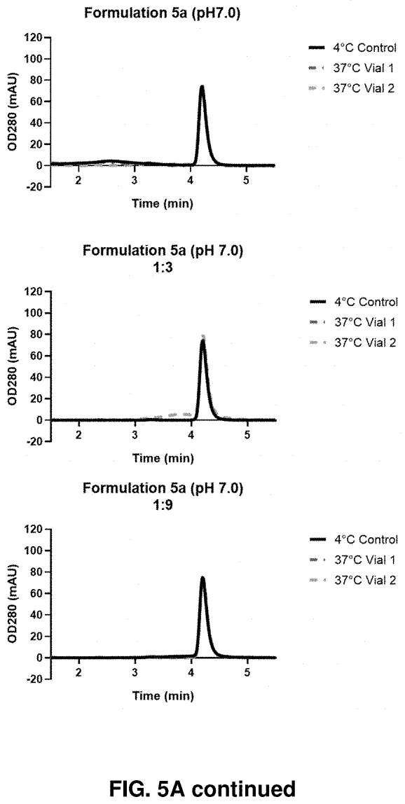

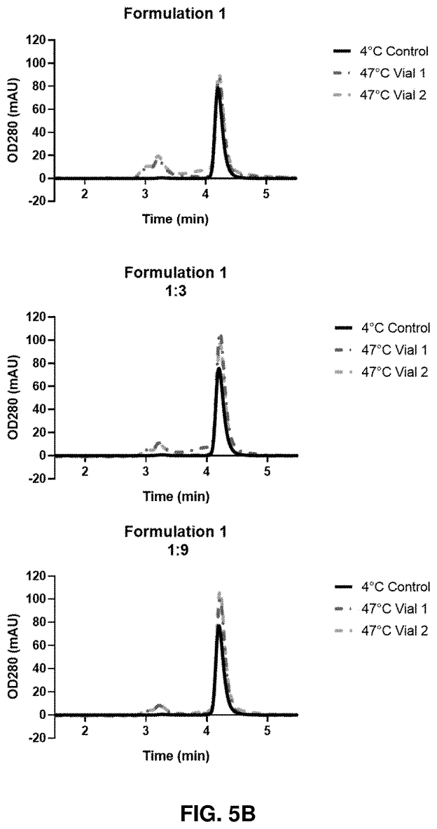

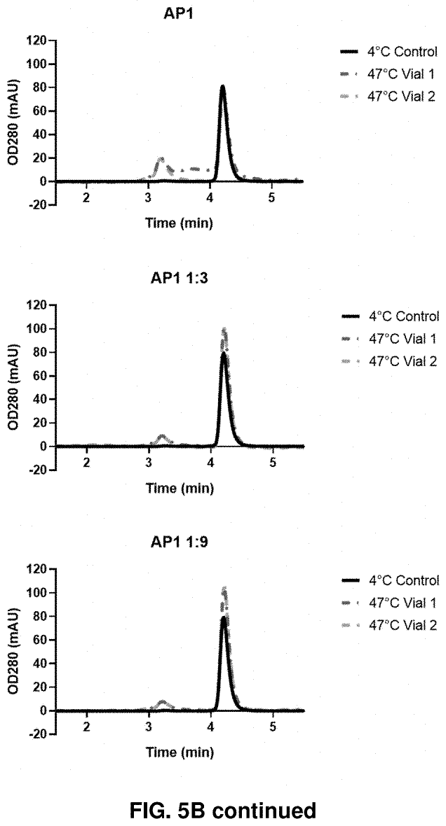

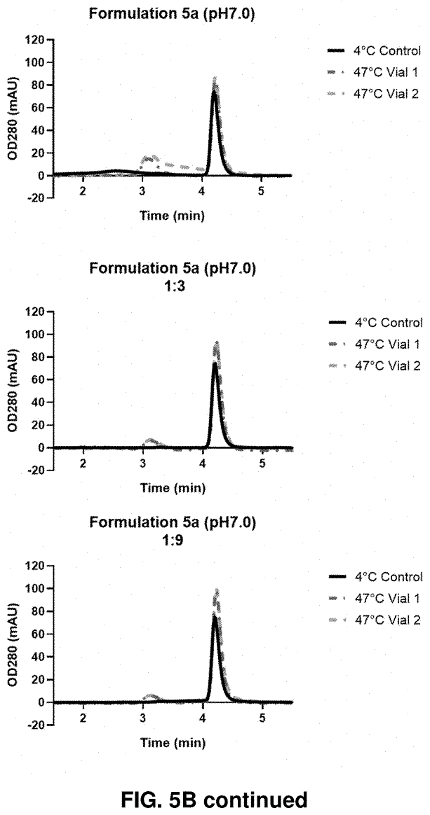

The RSV pre-fusion F (preF) protein of SEQ ID NO: 10 was dialyzed to different formulation buffers (AP1, formulation 1, and formulation 5a (pH7.0) and each composition was diluted to 0.3 mg/ml. Samples were prepared with and without compound III (final compound concentration of 5.2×10⁻⁶ M (trimer: compound ratio of:1:3), 1.6×10⁻⁵ (trimer: compound ratio of:1:9). Control samples were stored at 4° C. For heat stress, samples were kept for 6 weeks at 37 and 47° C. The trimer content was analyzed by analytical Size Exclusion Chromatography (SEC). At 37° C. trimer content was not changed compared to the control (FIG. 5A), whereas at 47° C. aggregation was observed (FIG. 5B). This aggregation was higher for the samples without compound compared to the samples with compound. This shows a protective effect of the compound on the pre-fusion protein for different formulation buffers.

### Example 7: Stabilizing Effect of Small Molecule Inhibitor on RSV Prefusion F Upon Ultrafiltration/Diafiltration (UF/DF)

UF/DF is a robust separation process based on size exclusion that finds application for a wide range of biotherapeutics. The RSV pre-fusion F (preF) protein (50 ml) of SEQ ID NO: 10 in a concentration of 0.6 mg/ml was ultrafiltrated/diafiltrated (UF/DF) to formulation 2 without PS20. The UF/DF was performed with and without compound. Compound IV (table 1) was added to the preF protein sample and to the UF/DF buffer in a 1:3 trimer:compound ratio. A kDa filter in the UF/DF (Cogent μscale TFF system (Merck Millipore, Burlington, MA)) was used and a diavolume of 350 ml UF/DF buffer was used.

After the UF/DF the hydrodynamic diameters were measured by Dynamic Light Scattering (DLS) UNcle from vendor Unchained Labs (Pleasanton, CA, US) two weeks after the UF/DF. After the UF/DF the diameter was 209.71 nm and 8.65 nm for the sample without and with compound respectively. Comparing the diameters to the starting material (hydrodynamic diameter of 10.93 nm) shows that the hydrodynamic diameter of the sample with compound is comparable to the starting material, whereas the sample without compound shows an increased diameter. This increased diameter is likely the result of beginning aggregation.

### Example 8: Vaccine Production and Manufacturing Improvement

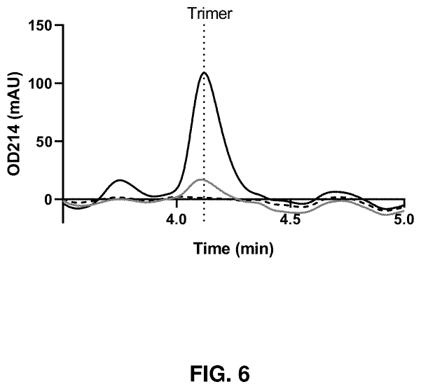

To investigate if addition of the stabilizing compound could also improve expression and yield of RSV F trimer, transfections of plasmids encoding soluble consensus subtype A RSV F protein (without stabilizing mutations) were performed with and without antiviral compound. Total trimer content after transfection was analysed by SEC and compared to transfection with a plasmid encoding stabilized preF. Transient transfection of stabilized preF (SEQ ID NO: 1) and unstabilized Consensus RSV A (SEQ ID NO: 2) were performed in 20 mL scale using HEK293F cells. 6 h after transfection compound III was added in a 1:3 (trimer:compound) ratio (compound concentration of 0.28 μM) to the unstabilized Consensus RSV A construct. After three days of protein production, cells were harvested and the trimer content of RSV F was evaluated in supernatant with analytical SEC (FIG. 6). No RSV F protein was detected for a transfection with unstabilized Consensus RSV A, however for the transfection of the same construct with compound III, expression of trimer could be detected. This indicates that the addition of the compound can be applied in the production and manufacturing process to increase RSV preF stability and yield.

### Example 9: HIV Env ConB-SOSIP Protein

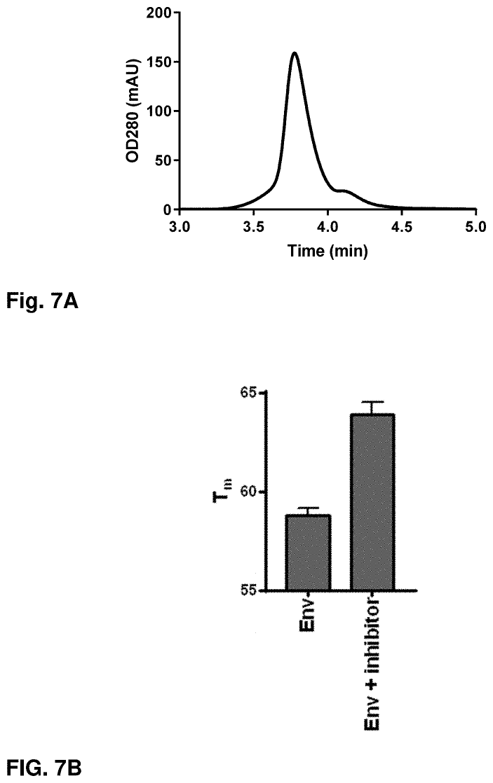

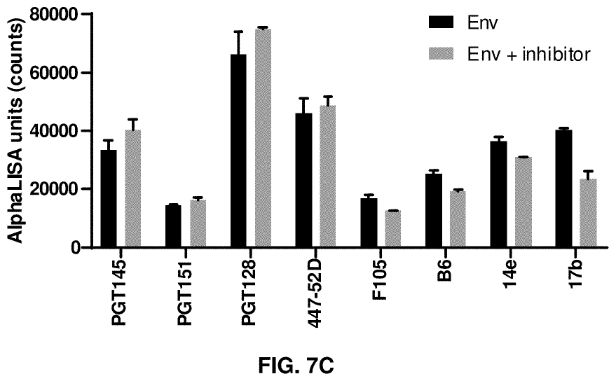

HIV-1 ConB-SOSIP (SEQ ID NO: 3) corresponds to the ectodomain of the HIV-1 surface protein gp140 of Glade B. The protein was produced by transient transfection of HEK293F cells and purified by lentil lectin and SEC as described (Rutten et. al., Cell Reports 2018). At the day of harvest the protein was trimeric as shown by analytical SEC-MALS (FIG. 7A). The purified pre-fusion trimeric HIV Env protein was stored at −80° C. until further analysis.

### Example 10: Thermo-Stability of HIV-1 ConB-SOSIP Env with and without Inhibitor

Thermostability was measured with nanoDSF using the UNcle from vendor Unchained Labs (Pleasanton, CA, US). HIV Env trimer was incubated at 0.8 mg/mi in Tris buffer (20 mM Citrate, 75 mM NaCl, 5% Sucrose, 0.03% Tween-80 pH 6.0) without or with a 50-fold molar excess of entry inhibitor compound XVII. Both solutions contained similar amounts of DMSO, which was 1.95% v/v). The inhibitor increased the melting temperature of the Env with ˜5° C. (FIG. 7B).

### Example 11: Storage Stability of ConB-SOSIP Env with and without Inhibitor

HIV env trimer was incubated in Citrate buffer (20 mM Citrate. 75 mM NaCl 5% Sucrose, 0.03% Tween-80 pH 6.0) at 0.8 mg/ml for 1 week at 4° C. with or without inhibitor. Entry inhibitor XVII was added in a 50-fold excess to the purified Env ConB SOSIP. As the inhibitor was dissolved in DMSO, the control sample with only Env contained the same concentration of DMSO (1.95% v/v) as the Env with the inhibitor. The quality of HIV Env can be evaluated with broadly neutralizing antibodies (bNAbs) that bind the closed, native pre-fusion conformation of Env, versus non-broadly neutralizing antibodies (non-bNAbs) which recognize non natively folded Envs (Rutten et. al., Cell Reports 18). The quality of HIV Env, as evaluated by the antigenicity measured with amplified luminescent proximity homogeneous assay (AlphaLISA), was superior for the Env sample that contained the entry inhibitor in the composition during 1 week storage (FIG. 7C). Preferential binding to bNAbs was higher and binding to all five tested non-bNAbs, except for 447-52D, was decreased for the composition that contained the inhibitor. This shows that the sample with inhibitor remained stable under these conditions and the protein without inhibitor showed decay and lost quality.

### Example 12: Influenza B HA Protein

### Protein Expression in Mammalian Cells

Transfections in Expi-CHO cells were performed with plasmids encoding Influenza B HA ectodomain. DNA fragments encoding the polypeptides were synthesized (Genscript; Piscataway, NJ) and cloned in the pcDNA2004 expression vector (modified pcDNA3 plasmid with an enhanced CMV promotor).

Influenza HA ectodomains fused to a C-terminal foldon trimerization domain were produced in ExpiCHO suspension cells (350 mL scale) cultured in ExpiCHO expression medium by transient transfection respective industrial grade DNA using ExpiFectamine transfection reagent (Gibco, ThermoFisher Scientific) according to the manufacturer's protocol. ExpiFectamine CHO Enhancer and ExpiCHO Feed (Gibco, ThermoFisher Scientific) were added to the cell cultures 1-day post transfection according to the manufacturer's protocol. ExpiCHO transfected cell suspensions were incubated at 32° C., 5% CO2 and the culture supernatants containing the secreted polypeptides were harvested between day 7-11. The culture supernatants were clarified by centrifugation, followed by filtration over a 0.2 μm bottle top filter (Corning).

From the harvested culture supernatants, the his-tagged polypeptides and respective wild type strains containing a Foldon trimerization domain were purified following a two-step protocol using an ÄKTA Avant 25 system (GE Healthcare Life Sciences). First, immobilized metal affinity chromatography was performed using a pre-packed cOmplete His-tag Purification Column (Roche), washed with 1 mM Imidazole and eluted with 300 mM Imidazole. Second, Size Exclusion Chromatography using a HiLoad Superdex 200 pg 26/600 Column (GE Healthcare Life Sciences) was performed. Trimer peak fractions were pooled and frozen for long term storage at −80° C. The purified HA B trimers UFV170091 (Yamagata lineage, Ectodomain of wild type HA of B/Massachussetts/2/12 fused to a foldon domain at the C-terminus; SEQ ID NO: 4) and UFV180300 (Victoria lineage, ectodomain of wild type HA of B/Colorado/06/2017_fused to foldon_SortA_v2(His) at the C-terminus, SEQ ID NO: 5) were stored at −80° C. until further analysis.

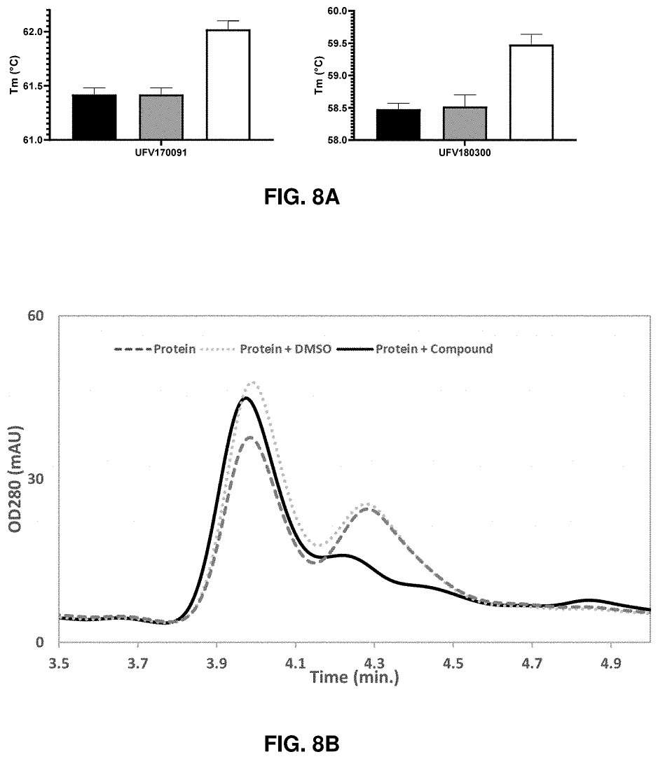

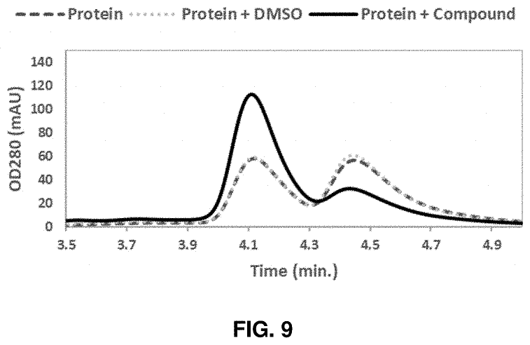

Melting temperature (Tm50) of both proteins with and without inhibitor (1:6 HA trimer:compound ratio) were measured using DSF (FIG. 8A). The inhibitor had a stabilizing effect since addition of the inhibitor resulted in an increase of the Tm of 0.5-1° C. for UFV170091 and UFV180300 respectively

Next, influenza HA ectodomain (based on HA of B/Brisbane/60/08, UFV180933, SEQ ID NO: 6) was transiently expressed in HEK293F cells with or without inhibitor (1:90 HA trimer:compound ratio). Supernatants were harvested 4 days after transfection and tested for the amount of monomer and trimer using analytical SEC analysis (FIG. 7B). Samples without inhibitor and with or without the diluent DMSO showed a high content of monomer. The sample with the fusion inhibitor showed hardly monomer and mostly trimer. Similar as for RSV and HIV, the influenza fusion inhibitor had a strong stabilizing effect on the native prefusion trimer. Therefore, also vaccine compositions with influenza HA type B native trimers can be stabilized during storage by addition of a non-therapeutic dose of fusion inhibitor.

### Example 13. Influenza A HA

Purified protein comprising the ectodomain of H1N1 A/Brisbane/59/2007 (SEQ ID NO: 7) was heat stressed for 6 weeks at 40 degree Celsius with and without compound XV. The trimer and monomer content were determined by analytical Size Exclusion Chromatography (SEC) (FIG. 8B). Compound XV was used in a 1:6 HA trimer:compound ratio. The addition of compound XV preserved the trimer content, whereas samples without compound XV, protein only and protein plus DMSO, showed more monomer and reduced trimer content. Therefore, also vaccine compositions with influenza HA type A native trimers can be stabilized during storage by addition of a non-therapeutic dose of fusion inhibitor.

### Example 14: Stabilizing Effect of Small Molecule Inhibitor on RSV Prefusion F in Different Formulation Buffers at Different Temperatures

The RSV pre-fusion F (preF) protein of SEQ ID NO: 10 was dialyzed to different formulation buffers (AP1, formulation 1, FB12, and formulation 5a (pH7.0) and each composition was diluted to 0.3 mg/ml. Samples were prepared with and without stabilizing compound III (final compound concentration of 5.2×10-6 M (trimer: compound ratio of 1:3) or 1.6×10-5M (trimer: compound ratio of 1:9). Control samples were stored at 4° C. Samples were kept for 9 or 16 or 26 weeks at 37° C. The trimer content was analyzed by analytical Size Exclusion Chromatography (SEC) and calculated relative to the 4° C. control.

### Results and Conclusion

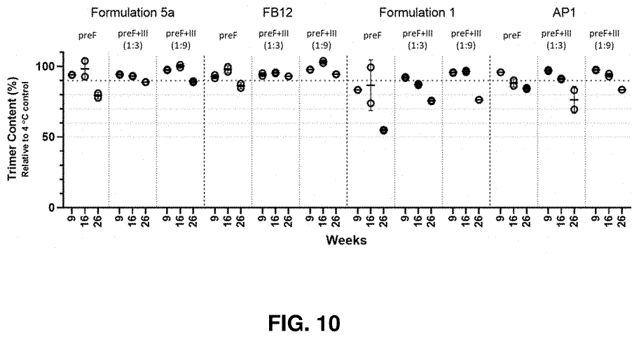

During incubation at 37° C., for periods up to 26 weeks, the addition of the stabilizing compound III protects from the formation of protein aggregates in liquid phase in different buffers (FIG. 10). Higher ratios (1:9) provided the best protection from aggregation over time.

Formulation 5a and FB12 seemed to be the most favorable buffers to keep the RSV preF protein stable in liquid phase at 37° C. Addition of the stabilizing compound III always improved the trimer content in these buffers. Formulation 1 and AP1 buffers showed the lowest protein content over time. Addition of the stabilizing compound III improved the trimer content in the Formulation 1 samples. The presence of the stabilizing compound III did not show protection from aggregation in AP1 samples.

### Example 15: Improved Trimer Stability after Removing Excess of Stabilizing Compound III with Dialysis

The RSV pre-fusion F (preF) protein of SEQ ID NO: 10 was dialyzed to different formulation buffers (AP1, formulation 1, FB12, and formulation 5a (pH7.0). Dialysis was performed at 4° C. in the dark using Slide-A-Lyzer™ G2 Dialysis Cassettes, 20K MWCO, 70 mL. 50 ml of protein was dialyzed against 10 L of buffer for 24 h. Protein was diluted in the formulation buffers to 0.3 mg/ml and stabilizing compound III was added in a 1:3 and 1:30 trimer: compound ratio and pre-incubated for 24 hours at 4° C. 0.75 ml of each formulation was filled in glass injection vials with rubber stopper and sealed with aluminum caps. Vials were frozen to −70° C. in 24 hours under controlled conditions. Samples were subsequently thawed to RT and analyzed by analytical Size Exclusion Chromatography (SEC) to measure loss of RSV F trimer compared the sample that was kept at 4° C. In addition, the dialyzed materials with and without stabilizing compound III were evaluated in DSF to measure the melting temperature. For experimental details see example 2.

### Results and Conclusion

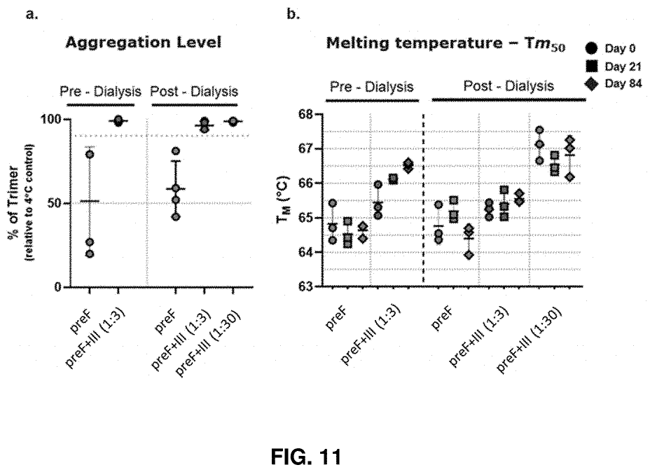

After slow freezing, the RSV preF protein tends to aggregate. Addition of stabilizing compound III protected from aggregation after slow freezing in pre- and post-dialysis samples (FIG. 11A). After dialysis and slow freezing, the presence of the stabilizing compound III prevented protein aggregation (higher than 90% of timer). Protection from aggregation was independent from the stabilizing compound III ratio added and there was no significant difference between a 1:3 and a 1:30 ratio in trimer percentage. Tm50 was higher in all samples where the stabilizing compound III was added (FIG. 11B). Addition of a higher stabilizing compound III ratio (1:30) yielded a higher Tm50 than a 1:3 ratio. The pre-dialysis samples with stabilizing compound III, showed a trend for an increased Tm50 over time. After dialysis, the Tm50 remained relatively constant in samples with stabilizing compound III over a prolonged period of time.

### Example 16: Improved Trimer Stability after Removing Access of Stabilizing Compound III with UF/DF

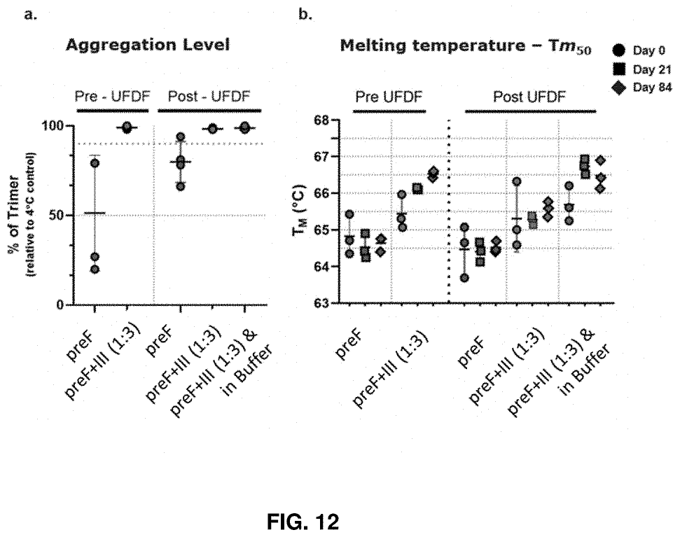

Buffer exchange of samples with and without stabilizing compound III was performed by ultrafiltration/diafiltration (UF/DF), using a Cogent μScale TFF system (Merck). The RSV pre-fusion F (preF) protein of SEQ ID NO: 10 was UF/DF-ed to Formulation buffer 6. Stabilizing compound III was preincubated with the sample and in one case also added to the buffer used for the UF/DF (see FIG. 12 labeled 1:3 & In Buffer). Settings of the UF/DF are summarized in Table 4. Runs were performed using Pelicon XL Biomax 50 cm2 filter cassettes. For each run, the system was flushed with MilliQ water at a crossflow of 30-50 mL/min, then the system was cleaned with NaOH 0.1M (recirculated for at least 5 min.) After this, the system was flushed again with MilliQ water until a pH of 7 was reached, followed by a 5 min. flush of the system with the desired buffer. Subsequently, the protein (50 mL) was added to the system for diafiltration against 7 diavolumes (350 mL) of the desired buffer.

TABLE 4
System settings during UF/DF

| | | |
|---|---|---|
| Crossflow | 30-35 | mL/min. |
| System Pump | 25 | % |
| Permeate flow | 10-14 | mL/min. |
| Permeate volume | 350 | mL/min. |
| P1 | 2.2 | bar |
| P2 | 0.2 | bar |
| DP | 2 | bar |
| TMP | 1.2 | bar |

Each formulation (pre and post UF/DF samples) was diluted to 0.3 mg/ml. 0.75 ml of each formulation was filled in glass injection vials with rubber stopper and sealed with aluminum caps. Vials were frozen to −70° C. in 24 hours under controlled conditions. Samples were subsequently thawed to RT and analyzed by analytical Size Exclusion Chromatography (SEC) to measure loss of RSV F trimer compared the sample that was kept at 4° C. In addition, the different formulations with and without stabilizing compound III were evaluated in DSF to measure the melting temperature. For experimental details see example 2.

### Results and Conclusion

UF/DF procedure under current conditions did not result in aggregation. After UF/DF and slow freezing, all samples containing stabilizing compound III were protected from aggregation irrespective of the stabilizing compound III concentration (FIG. 12A). Slow freezing in the preUF/DF buffer yielded more aggregates than slow freezing in a Formulation 6 buffer. Samples containing FI (without buffer exchange or with FI in the exchange buffer) showed a trend to yield an increased Tm₅₀ over time (FIG. 12B). Samples that included the stabilizing compound III in the protein and in the diavolume yielded the highest Tm₅₀.

### Example 17: Advantage of High Ratios of Stabilizing Compound III and Total Stabilizing Compound III Bound to preF

The RSV pre-fusion F (preF) protein of SEQ ID NO: 10 was dialyzed (see example 15 for details on the method) or UF/DF (see example 16 for details on the method) to different formulation buffers (Formulation 1 without PS20, Formulation 2 without PS20 and AP1). Stabilizing compound III was added in a 1:50 trimer:compound ratio before the UD/DF and incubated for 24 hours at 4° C. The excess of unbound stabilizing compound III is removed during the UF/DF process.

Each formulation (pre and post dialysis samples) was diluted to 0.3 mg/ml. 0.75 ml of each formulation was filled in glass injection vials with rubber stopper and sealed with aluminum caps. Vials were slowly frozen to −70° C. in 24 hours. Samples were subsequently thawed to RT and analyzed by analytical Size Exclusion Chromatography (SEC) to measure loss of RSV F trimer compared the sample that was kept at 4° C. In addition, the different formulations with and without compound III were evaluated in DSF to measure the melting temperature. For experimental details see example 2.

Additionally, the concentration of stabilizing compound III present in the samples after UF/DF was determined by LC-MS/MS.

### Results and Conclusion

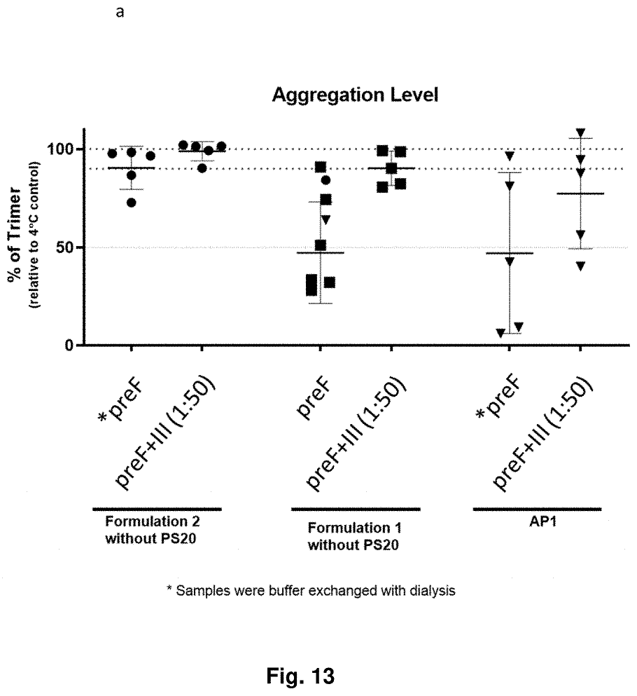

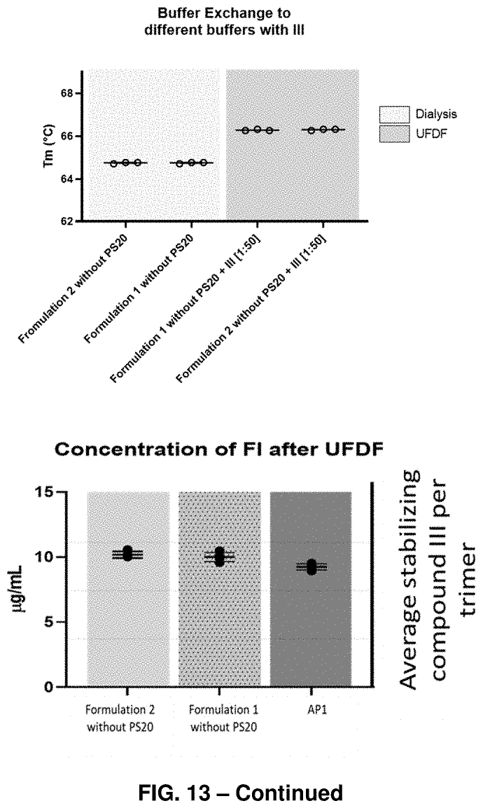

Irrespective of the buffer, the addition of the stabilizing compound III protected from aggregation after buffer exchange and subsequent slow freezing (FIG. 13A). Even for the non-optimal Formulation 1 without PS20, the stabilizing compound III provided protection against aggregation. AP1 buffer is not optimal and the stabilizing compound III did not provide enough protections against aggregation.

Addition of stabilizing compound III increased the Tm50 irrespective of the buffer (FIG. 13B). After UF/DF, the stabilizing compound III remained bound to the protein. Adding a high excess of stabilizing compound III (1:50) enabled binding (and most likely trapping) of ˜2.6 molecules of stabilizing compound III per trimer (FIG. 13C). Data correlates with DSF analysis depicting a higher/improved Tm50 when ratios above 1:3 were added (FIGS. 11B and 12B).

### Example 18: Immunogenicity in Mice

Balb/c mice (6-8 weeks old, female) were given two intramuscular (i.m.) immunizations 28 days apart with increasing doses of preF protein (SEQ NO 10) (1.5, 5 or 15 μg), or preF protein (SEQ ID NO 10) (1.5, 5 or 15 μg), combined with a 3-fold, 10-fold or 30-fold molar excess of stabilizing compound III based on the preF trimer. Forty-two days post first dosing, serum samples were collected and analyzed for virus neutralization titers using an automated firefly luciferase assay (FFL-VNA) that measures inhibition of infection of the RSV-CL57 strain on A549 cells.

VNA titers of the preF groups formulated with stabilizing compound III were compared across doses for non-inferiority test with a 4-fold margin (Tobit model with Bonferroni correction for multiple comparisons) with preF without fusion inhibitor as benchmark.

### Results and Conclusion

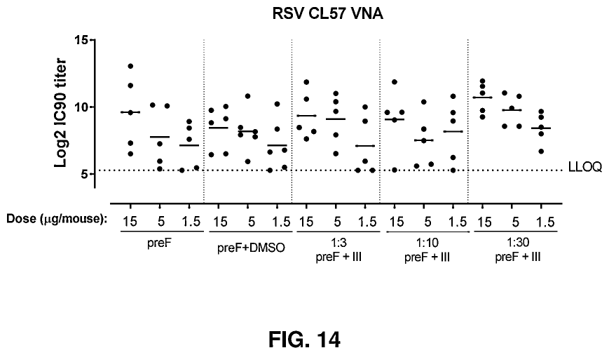

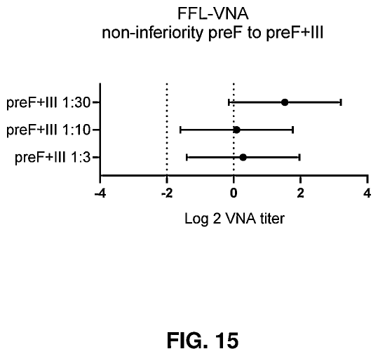

VNA titers induced by PreF combined with stabilizing compound III, independent of the amount of stabilizing compound III added, were not lower compared to titers induced by preF without the addition of stabilizing compound III based on the FFL-VNA responses measured at day 42 (FIG. 14). The immunogenicity of preF protein vaccine candidate was not negatively affected by the addition of stabilizing compound III at the tested ratios based on the non-inferiority analysis with a 4-fold margin (FIG. 15).

### Example 19: RSV Neutralization in Human Airway Epithelial Cells

Pooled sera from Example 18 were used to perform a VNA on differentiated primary human Airway Epithelial Cells (hAEC) grown at an air-liquid interface and which mimic the human upper respiratory tract.

The hAEC transwell inserts are prepared at Epithelix (Switzerland). In short, primary human cells from a pool of 14 healthy human donors are cultured at an air-liquid interface for >3 weeks to differentiate into a complex tissue that consists of basal, goblet and ciliated cells and which is covered by a mucus layer. The hAEC inserts are especially rich in ciliated cells, the natural in vivo target of RSV. The neutralization assay was performed using an RSV-A2 reporter (GFP) virus. The level of virus infection was determined by visualizing the GFP signal using a Cytation 1 automated microscope (BioTek, USA) 4 days post infection. Infection is depicted in grayscale (i.e. infected cells in light gray).

### Results and Conclusion

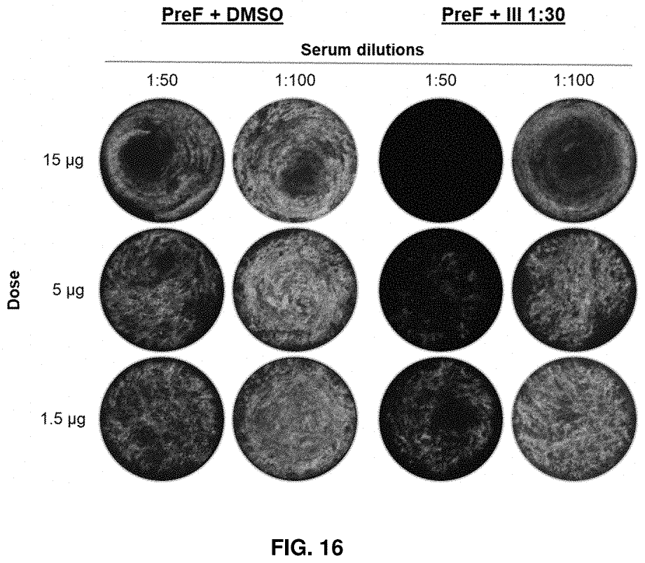

Sera pool of preF with stabilizing compound III (1:30) showed higher neutralization titers as compared to preF+DMSO in the differentiated human airway epithelial cells (hAEC) at 1/50 dilution at all three dosages of preF (FIG. 16).

SEQUENCES
SEQ ID NO: 1: Stabilized RSV F protein (PreF) fused to foldon
domain (underlined) (p27 peptide underlined and bold)
MELLILKANAITTILTAVTFCFASGQNITEEFYQSTCSAVSKGYLSALRTGWYTSVITIELS
NIKEIKCNGTDAKVKLIKQELDKYKNAVTELQLLMQSTPATNNRARRELPRFMNYTLN
NAKKTNVTLSKKRKRRFLGFLLGVGSAIASGVAVSKVLHLEGEVNKIKSALLSTNKAV
VSLSNGVSVLTSKVLDLKNYIDKQLLPIVNKQSCSIPNIETVIEFQQKNNRLLEITREFSVN
AGVTTPVSTYMLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVSFFPQAETCK
VQSNRVFCDTMNSLTLPSEVNLCNVDIFNPKYDCKIMTSKTDVSSSVITSLGAIVSCYGK
TKCTASNKNRGIIKTFSNGCDYVSNKGVDTVSVGNTLYYVNKQEGKSLYVKGEPIINFY
DPLVFPSNEFDASISQVNEKINQSLAFIRKSDELLSAIGGYIPEAPRDGQAYVRKDGEWVL
LSTFL
SEQ ID NO: 10: Soluble, processed, stabilized RSV F protein
(PreF) fused to foldon domain (underlined)
QNITEEFYQSTCSAVSKGYLSALRTGWYTSVITIELSNIKEIKCNGTDAKVKLIKQELDKY
KNAVTELQLLMQSTPATNNRAFLGFLLGVGSAIASGVAVSKVLHLEGEVNKIKSALLST
NKAVVSLSNGVSVLTSKVLDLKNYIDKQLLPIVNKQSCSIPNIETVIEFQQKNNRLLEITR
EFSVNAGVTTPVSTYMLTNSELLSLINDMPITNDQKKLMSNNVQIVRQQSYSIMSIIKEEV
LAYVVQLPLYGVIDTPCWKLHTSPLCTTNTKEGSNICLTRTDRGWYCDNAGSVSFFPQA
ETCKVQSNRVFCDTMNSLTLPSEVNLCNVDIFNPKYDCKIMTSKTDVSSSVITSLGAIVS
CYGKTKCTASNKNRGIIKTFSNGCDYVSNKGVDTVSVGNTLYYVNKQEGKSLYVKGEP
IINFYDPLVFPSNEFDASISQVNEKINQSLAFIRKSDELLSAIGGYIPEAPRDGQAYVRKDG
EWVLLSTFL
SEQ_ID_NO: 2: Consensus RSV F A fused to foldon domain
(underlined)
MELLILKANAITTILTAVTFCFASGQNITEEFYQSTCSAVSKGYLSALRTGWYTSVITIELS
NIKENKCNGTDAKVKLIKQELDKYKNAVTELQLLMQSTPAANNRARRELPRFMNYTLN
NAKKTNVTLSKKRKRRFLGFLLGVGSAIASGIAVSKVLHLEGEVNKIKSALLSTNKAVV
SLSNGVSVLTSKVLDLKNYIDKQLLPIVNKQSCSISNIETVIEFQQKNNRLLEITREFSVNA
GVTTPVSTYMLTNSELLSLINDMPITNDQKKLMSNNVQIVRQQSYSIMSIIKEEVLAYVV
QLPLYGVIDTPCWKLHTSPLCTTNTKEGSNICLTRTDRGWYCDNAGSVSFFPQAETCKV
QSNRVFCDTMNSLTLPSEVNLCNVDIFNPKYDCKIMTSKTDVSSSVITSLGAIVSCYGKT
KCTASNKNRGIIKTFSNGCDYVSNKGVDTVSVGNTLYYVNKQEGKSLYVKGEPIINFYD
PLVFPSDEFDASISQVNEKINQSLAFIRKSDELLSAIGGYIPEAPRDGQAYVRKDGEWVLL
STFL
SEQ ID NO: 3 ConB_SOSIP
MRVKGIRKNYQHLWRWGTMLLGMLMICSAAEKLWVTVYYGVPVWKEATTTLFCASD
AKAYDTEVHNVWATHACVPTDPNPQEVVLENVTENFNMWKNNMVEQMHEDIISLWD
QSLKPCVKLTPLCVTLNCTDLNNNTTNNNSSSEKMEKGEIKNCSFNITTSIRDKVQKEYA
LFYKLDVVPIDNNNTSYRLISCNTSVITQACPKVSFEPIPIHYCAPAGFAILKCNDKKFNG
TGPCTNVSTVQCTHGIRPVVSTQLLLNGSLAEEEVVIRSENFTDNAKTIIVQLNESVEINC
TRPNNNTRKSIHIGPGRAFYATGDIIGDIRQAHCNISRTKWNNTLKQIVKKLREQFGNKTI
VFNQSSGGDPEIVMHSFNCGGEFFYCNTTQLFNSTWNSNGTWNNTTGNDTITLPCRIKQI
INMWQEVGKAMYAPPIRGQIRCSSNITGLLLTRDGGNNNNNTTETFRPGGGDMRDNWR
SELYKYKVVKIEPLGVAPTKCKRRVVQRRRRRRAVGIGAMFLGFLGAAGSTMGAASIT
LTVQARQLLSGIVQQQNNLLRAPEAQQHLLQLTVWGIKQLQARVLAVERYLKDQQLLG
IWGCSGKLICCTAVPWNTSWSNKSLDEIWDNMTWMQWEREIDNYTGLIYTLIEESQNQ
QEKNEQELLELD
SEQ ID NO: 4: UFV170091, HA ectodomain (based on B/
Massachusetts/2/12) fused to foldon domain (underlined) and
with Histag
MKAIIVLLMVVTSNADRICTGITSSNSPHVVKTATQGEVNVTGVIPLTTTPTKSYFANLK
GTKTRGKLCPDCLNCTDLDVALGRPMCVGTTPSAKASILHEVRPVTSGCFPIMHDRTKI
RQLANLLRGYENIRLSTQNVIDAEKAPGGPYRLGTSGSCPNATSKSGFFATMAWAVPKD
NNKNATNPLTVEVPYICAEGEDQITVWGFHSDDKTQMKNLYGDSNPQKFTSSANGVTT
HYVSQIGGFPDQTEDGGLPQSGRIVVDYMMQKPGKTGTIVYQRGVLLPQKVWCASGRS
KVIKGSLPLIGEADCLHEKYGGLNKSKPYYTGEHAKAIGNCPIWVKTPLKLANGTKYRP
PAKLLKERGFFGAIAGFLEGGWEGMIAGWHGYTSHGAHGVAVAADLKSTQEAINKITK
NLNSLSELEVKNLQRLSGAMDELHNEILELDEKVDDLRADTISSQIELAVLLSNEGIINSE
DEHLLALERKLKKMLGPSAVDIGNGCFETKHKCNQTCLDRIAAGTFNAGEFSLPTFDSL
NITASGSLVPRGSGSGYIPEAPRDGQAYVRKDGEWVLLSTFLGGSIEGRGGSHHHHHH
SEQ ID NO: 5: UFV180300, HA ectodomain (based on B/Colorado/
06/2017) fused to foldon domain (underlined) with Histag
MKAIIVLLMVVTSSADRICTGITSSNSPHVVKTATQGEVNVTGVIPLTTTPTKSHFANLKG
TETRGKLCPKCLNCTDLDVALGRPKCTGKIPSARVSILHEVRPVTSGCFPIMHDRTKIRQ
LPNLLRGYEHVRLSTHNVINAEGAPGGPYKIGTSGSCPNITNGNGFFATMAWAVPDKNK
TATNPLTIEVPYVCTEGEDQITVWGFHSDNETQMAKLYGDSKPQKFTSSANGVTTHYVS
QIGGFPNQTEDGGLPQSGRIVVDYMVQKSGKTGTITYQRGILLPQKVWCASGRSKVIKG
SLPLIGEADCLHEKYGGLNKSKPYYTGEHAKAIGNCPIWVKTPLKLANGTKYRPPAKLL
KERGFFGAIAGFLEGGWEGMIAGWHGYTSHGAHGVAVAADLKSTQEAINKITKNLNSL
SELEVKNLQRLSGAMDELHNEILELDEKVDDLRADTISSQIELAVLLSNEGIINSEDEHLL
ALERKLKKMLGPSAVEIGNGCFETKHKCNQTCLDKIAAGTFDAGEFSLPTFDSLNITASG
SLVPSGSPGSGYIPEAPRDGQAYVRKDGEWVLLSTFLGGSLPETGGGSHHHHHH
SEQ ID NO: 6: UFV180933, HA ectodomain of B/Brisbane/60/08
with C-tag
MKAIIVLLMVVTSNADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIR
QLPNLLRGYEHIRLSTHNVINAENAPGGPYKIGTSGSCPNITNGNGFFATMAWAVPKND
KNKTATNPLTIEVPYICTEGEDQITVWGFHSDNETQMAKLYGDSKPQKFTSSANGVTTH
YVSQIGGFPNQTEDGGLPESGRIVVDYMVQKSGKTGTITYQRGILLPQKVWCASGRSKV
IKGSLPLIGEADCLHEKYGGLNKSKPYYTGEHAKAIGNCPIWVKTPLKLANGTKYRPPA
KLLKEQGFFGAIAGFLEGGWEGMIAGWHGYTSHGAHGVAVAADLKSTQEAINKITKNL
NSLSELEVKNLQRLSGAMDELHNEILELDEKVDDLRADTISSQIELAVLLSNEGIINSEDE
HLLALERKLKKMLGPSAVEIGNGCFETKHKCNQTCLDRIAAGTFDAGEFSLPTFDSLNIT
AEPEA
SEQ ID NO: 7: ecto domain of H1N1 A/Brisbane/59/2007, with
Histag
MKVKLLVLLCTFTATYADTICIGYHANNSTDTVDTVLEKNVTVTHSVNLLENSHNGKL
CLLKGIAPLQLGNCSVAGWILGNPECELLISKESWSYIVEKPNPENGTCYPGHFADYEEL
REQLSSVSSFERFEIFPKESSWPNHTVTGVSASCSHNGESSFYRNLLWLTGKNGLYPNLS
KSYANNKEKEVLVLWGVHHPPNIGDQKALYHTENAYVSVVSSHYSRKFTPEIAKRPKV
RDQEGRINYYWTLLEPGDTIIFEANGNLIAPRYAFALSRGFGSGIINSNAPMDKCDAKCQ
TPQGAINSSLPFQNVHPVTIGECPKYVRSAKLRMVTGLRNIPSIQSQGLFGAIAGFIEGGW
TGMVDGWYGYHHQNEQGSGYAADQKSTQNAINGITNKVNSVIEKMNTQFTAVGKEFN
KLERRMENLNKKVDDGFIDIWTYNAELLVLLENERTLDFHDSNVKNLYEKVKSQLKNN
AKEIGNGCFEFYHKCNDECMESVKNGTYDYPKYSEESKLNREKIDGVKLESMGVYQIE
GRHHHHHHH
CR9506 heavy chain (SEQ ID NO: 8)
EVQLVQSGAEVKKPGSSVKVSCKASGGTFSRSLITWVRQAPGQGLEWMGEISLVFGSAKNAQKF
QGRVTITADESTSTAHMEMISLKHEDTAVYYCAAHQYGSGTHNNFWDESELRFDLWGQGTLVT
VSSASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSVMHEALHNH
YTQKSLSLSPGK
CR9506 light chain (SEQ ID NO: 9)
DIVMTQSPSSLSASVGDRVTIACRASQSIGTYLNWYQQKPGKAPKLLIYAASSLQSGVPSRFSGS
GSGTHFTLAISSLQAEDFATYSCQQSYTIPYTFGQGTKLEIKRTVAAPSVFIFPPSDEQLKSGTA
SVVCLLNNFYPREAKVQWKVDNALQSGNSQESVTEQDSKDSTYSLSSTLTLSKADYEKHKVYACE
VTHQGLSSPVTKSFNRGEC

1. A vaccine composition comprising an immunologically effective amount of a viral fusion protein antigen and a stabilizing amount of an antiviral compound.

2. The vaccine composition according to claim 1, wherein the viral fusion protein is a class I fusion protein.

3. The vaccine composition according to claim 1, wherein the viral fusion protein antigen is a metastable class I fusion protein.

4. The vaccine composition according to claim 1, wherein the viral fusion protein is a trimeric class I fusion protein.

5. The vaccine composition according to claim 1, wherein the antiviral compound is a fusion inhibitor or a viral entry inhibitor.

6. The vaccine composition according to claim 1, wherein the stabilizing amount of the antiviral compound is a sub-therapeutically effective amount of said antiviral compound.

7. The vaccine composition according to claim 6, wherein the stabilizing amount of the antiviral compound is at least 100× lower that the therapeutically effective amount of said antiviral compound.

8. The vaccine composition according claim 1, wherein the viral fusion protein and said antiviral compound are present in a trimer:compound ratio ranging between and including 1:1 and 1:300.

9. The vaccine composition according to claim 1, wherein the viral fusion protein is an RSV fusion (F) protein.

10. The vaccine composition according to claim 9, wherein the RSV F protein is a pre-fusion RSV F protein.

11. The vaccine composition according to claim 8, wherein the antiviral compound is an RSV fusion or entry inhibitor.

12. The vaccine composition according to claim 11, wherein the RSV fusion or entry inhibitor is selected from the group consisting of compounds I-XVI in Table 1 and suitable analogues thereof.

13. The vaccine composition according to claim 1, wherein the fusion protein is an HIV envelope (env) protein.

14. The vaccine composition according to claim 13, wherein the HIV env protein is a pre-fusion HIV env protein.

15. The vaccine composition according to claim 13, wherein antiviral compound is an HIV fusion or entry inhibitor.

16. The vaccine composition according to claim 15, wherein the HIV fusion or entry inhibitor is selected from the group consisting of the compounds XVII-XXIII in Table 2, and suitable analogues thereof.

17. The vaccine composition according to claim 1, wherein the fusion protein is an influenza A hemagglutinin (HA) protein.

18. The vaccine composition according to claim 17, wherein the influenza HA protein is an influenza B HA protein.

19. The vaccine composition according to claim 17, wherein the antiviral compound is an influenza fusion or entry inhibitor.

20. The vaccine composition according to claim 17, wherein the antiviral compound is selected from the group consisting of the compounds XXIV-XXVI in Table 3, and suitable analogues thereof.

21. The vaccine composition according to claim 18, wherein the antiviral compound is compound XXVII in Table 3 or a suitable analogue thereof.

22. The vaccine composition according to claim 1, wherein said composition is a liquid composition.

23. The vaccine composition according to claim 1, which has an improved stability upon storage at a temperature ranging between room temperature and 47° C. for at least 6 weeks.

24. The vaccine composition according to claim 1, which has an improved stability after freezing until at −80° C. and subsequent thawing of said composition.

25. The vaccine composition according to claim 1, which has an improved stability upon ultrafiltration/diafiltration.

26. A methods for preparing the vaccine composition according to claim 1, said method comprising admixing an immunologically effective amount of said viral fusion protein antigen with a stabilizing amount of the antiviral compound.

27. A method for reducing aggregation of a viral fusion protein in the vaccine composition according to claim 1, comprising admixing an immunologically effective amount of the fusion protein antigen with a stabilizing amount of the antiviral compound.

28. A method for reducing aggregation of a viral fusion protein in a vaccine composition after freeze-thawing of said vaccine composition, comprising preparing the vaccine composition by admixing an immunologically effective amount of a fusion protein antigen with a stabilizing amount of an antiviral compound.

29. A method of preserving a vaccine comprising a viral fusion protein antigen, which method comprises preparing a vaccine composition according to claim 1.

30. A method of preserving a vaccine comprising a viral fusion protein antigen, comprising preparing a vaccine composition according to claim 1 and storing said composition at a temperature ranging between 2-8° C. for at least 24 months.

31. A method for stably maintaining a liquid vaccine composition comprising a viral fusion protein antigen, the method comprising preparing a vaccine composition according to claim 1 and storing said composition at a temperature ranging between 2-8° C. for at least 24 months.

32. A method of producing a (thermo)stable viral fusion protein immunogen, comprising expressing the viral fusion protein immunogen in the presence of a stabilizing amount of an antiviral compound.


SEQUENCE LISTING (10 sequences)

SEQ ID NO: 1 - protein, 544 aa, Artificial Sequence
  Stabilized RSV F protein (PreF) fused to foldon domain
MELLILKANAITTILTAVTFCFASGQNITEEFYQSTCSAVSKGYLSALRTGWYTSVITIELSNIKEIKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKSDELLSAIGGYIPEAPRDGQAYVRKDGEWVLLSTFL

SEQ ID NO: 2 - protein, 544 aa, Artificial Sequence
  Consensus RSV F A fused to foldon domain
MELLILKANAITTILTAVTFCFASGQNITEEFYQSTCSAVSKGYLSALRTGWYTSVITIELSNIKENKCNGTDAKVKLIKQELDKYKNAVTELQLLMQSTPAANNRARRELPRFMNYTLNNAKKTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 3 - protein, 659 aa, Artificial Sequence
  ConB_SOSIP
MRVKGIRKNYQHLWRWGTMLLGMLMICSAAEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLNNNTTNNNSSSEKMEKGEIKNCSFNITTSIRDKVQKEYALFYKLDVVPIDNNNTSYRLISCNTSVITQACPKVSFEPIPIHYCAPAGFAILKCNDKKFNGTGPCTNVSTVQCTHGIRPVVSTQLLLNGSLAEEEVVIRSENFTDNAKTIIVQLNESVEINCTRPNNNTRKSIHIGPGRAFYATGDIIGDIRQAHCNISRTKWNNTLKQIVKKLREQFGNKTIVFNQSSGGDPEIVMHSFNCGGEFFYCNTTQLFNSTWNSNGTWNNTTGNDTITLPCRIKQIINMWQEVGKAMYAPPIRGQIRCSSNITGLLLTRDGGNNNNNTTETFRPGGGDMRDNWRSELYKYKVVKIEPLGVAPTKCKRRVVQRRRRRRAVGIGAMFLGFLGAAGSTMGAASITLTVQARQLLSGIVQQQNNLLRAPEAQQHLLQLTVWGIKQLQARVLAVERYLKDQQLLGIWGCSGKLICCTAVPWNTSWSNKSLDEIWDNMTWMQWEREIDNYTGLIYTLIEESQNQQEKNEQELLELD

SEQ ID NO: 4 - protein, 589 aa, Artificial Sequence
  HA ectodomain (based on B/Massachusetts/2/12) fused to foldon domain with Histag
MKAIIVLLMVVTSNADRICTGITSSNSPHVVKTATQGEVNVTGVIPLTTTPTKSYFANLKGTKTRGKLCPDCLNCTDLDVALGRPMCVGTTPSAKASILHEVRPVTSGCFPIMHDRTKIRQLANLLRGYENIRLSTQNVIDAEKAPGGPYRLGTSGSCPNATSKSGFFATMAWAVPKDNNKNATNPLTVEVPYICAEGEDQITVWGFHSDDKTQMKNLYGDSNPQKFTSSANGVTTHYVSQIGGFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITASGSLVPRGSGSGYIPEAPRDGQAYVRKDGEWVLLSTFLGGSIEGRGGSHHHHHH

SEQ ID NO: 5 - protein, 590 aa, Artificial Sequence
  HA ectodomain (based on B/Colorado/06/2017) fused to foldon domain with Histag
MKAIIVLLMVVTSSADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEGAPGGPYKIGTSGSCPNITNGNGFFATMAWAVPDKNKTATNPLTIEVPYVCTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDKIAAGTFDAGEFSLPTFDSLNITASGSLVPSGSPGSGYIPEAPRDGQAYVRKDGEWVLLSTFLGGSLPETGGGSHHHHHH

SEQ ID NO: 6 - protein, 540 aa, Artificial Sequence
  UFV180933, HA ectodomain of B/Brisbane/60/08 with C-tag
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHIRLSTHNVINAENAPGGPYKIGTSGSCPNITNGNGFFATMAWAVPKNDKNKTATNPLTIEVPYICTEGEDQITVWGFHSDNETQMAKLYGDSKPQKFTSSANGVTTHYVSQIGGFPNQTEDGGLPESGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKEQGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAEPEA

SEQ ID NO: 7 - protein, 539 aa, Artificial Sequence
  ecto domain of H1N1 A/Brisbane/59/2007, with Histag
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGDQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSQGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQIEGRHHHHHHH

SEQ ID NO: 8 - protein, 460 aa, Artificial Sequence
  CR9506 heavy chain
EVQLVQSGAEVKKPGSSVKVSCKASGGTFSRSLITWVRQAPGQGLEWMGEISLVFGSAKNAQKFQGRVTITADESTSTAHMEMISLKHEDTAVYYCAAHQYGSGTHNNFWDESELRFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, Artificial Sequence
  CR9506 light chain
DIVMTQSPSSLSASVGDRVTIACRASQSIGTYLNWYQQKPGKAPKLLIYAASSLQSGVPSRFSGSGSGTHFTLAISSLQAEDFATYSCQQSYTIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 490 aa, Artificial Sequence
  Soluble, processed, stabilized RSV F protein (PreF) fused to foldon domain
QNITEEFYQSTCSAVSKGYLSALRTGWYTSVITIELSNIKEIKCNGTDAKVKLIKQELDKYKNAVTELQLLMQSTPATNNRAFLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSNEFDASISQVNEKINQSLAFIRKSDELLSAIGGYIPEAPRDGQAYVRKDGEWVLLSTFL